(19) United States
(12) Patent Application Publication
(10) Pub. No.: US 2024/0368063 A1
(43) Pub. Date: Nov. 7, 2024

(54) PREPARATION METHOD, INTERMEDIATE COMPOUNDS, AND SYNTHESIS METHOD FOR A CLASS OF ANTHRACENE-DERIVED TOXINS IN ORGANIC CHEMISTRY

(71) Applicant: WUYAN PHARMACEUTICAL TECHNOLOGY (SHANGHAI) CO., LTD, Shanghai (CN)

(72) Inventors: Dongcheng Qiu, Shanghai (CN); Shunhui He, Shanghai (CN)

(73) Assignee: WUYAN PHARMACEUTICAL TECHNOLOGY (SHANGHAI) CO., LTD, Shanghai (CN)

(21) Appl. No.: 18/774,975

(22) Filed: Jul. 17, 2024

(30) Foreign Application Priority Data
Oct. 17, 2022 (CN) 202211323072.0

(51) Int. Cl. C07C 49/16; C07C 303/02; C07C 303/28

(52) U.S. Cl. CPC C07C 49/16; C07C 303/02; C07C 303/28

(57) ABSTRACT

This invention relates to the field of organic synthesis technology, specifically an intermediate compound for the preparation of a class of anthracene ring toxins derivatives. Starting from relatively inexpensive and readily available anthracene drugs or their derivatives (IV), an intermediate compound (III) has been cleverly designed, which, through a single-step reaction, can be converted into the corresponding pyran, oxazole, and thiazole-like compounds (V); this effectively addresses the issues of restricted raw materials and high costs, as these commercially available raw materials are inexpensive and abundantly supplied in the market, reducing the raw material costs for producing compound (V) and ensuring a stable supply of raw materials for large-scale production.

### CROSS-REFERENCE TO RELATED APPLICATIONS

This application claims priority to Chinese Patent Application Ser. No. CN202211323072.0 filed on 17 Oct. 2022.

### TECHNOLOGY FIELD

This invention relates to the field of organic synthesis technology, specifically a method for the preparation of a class of anthracene ring-containing toxin derivatives, intermediate compounds, and synthetic methods.

### BACKGROUND TECHNOLOGY

Antibody drug conjugates (ADCs) are a novel class of anti-tumor drugs, which involve linking cytotoxic agents to antibodies. By recognizing specific antigens on the surface of cancer cells, the antibodies facilitate the internalization of the cytotoxic agents into the cancer cells, thereby delivering the cytotoxic payload to the target site and achieving targeted therapy for malignant tumors.

Compared with traditional small molecule anti-cancer drugs, ADCs have greater specificity and efficacy due to their ability to target recognition with antibodies and high activity of toxins. ADCs consist of three different components, namely antibodies, linkers, and cytotoxins. Antibodies achieve targeting, linkers ensure stability during ADC transport in the blood, and once reaching the target site, toxins exert their killing effect on cancer cells.

Anthracene-based drugs have excellent cytotoxicity and are ideal cytotoxins in ADC synthesis. Scientists discovered a highly active metabolite, PNU-159682, during the anti-tumor research of nemorubicin, a derivative of anthracycline with anti-tumor activity. PNU-159682 is a DNA topoisomerase II (Topo II) inhibitor with excellent cytotoxicity. In ADC synthesis, PNU-159682 is a more effective and tolerable ADC cytotoxin compared to anthracyclines. Recent studies on anthracene-based drugs such as nemorubicin and its main metabolite PNU-159682 have shown that these drugs can overcome the limitations of multidrug resistance and cardiotoxicity associated with doxorubicin. Furthermore, compared to anthracyclines, PNU-159682 exhibits three orders of magnitude higher cytotoxicity against various tumor cell lines, including cells resistant to anthracyclines. Studies have shown that PNU-159682 inhibits a range of human tumor cell lines with IC70 values ranging from 0.07 to 0.58 nM, demonstrating 2,360 to 790 times higher potency compared to nemorubicin and 6,420 to 2,100 times higher potency compared to doxorubicin. PNU-159682 weakly inhibits the activity of topoisomerase II at a concentration of 100 μM. The chemical structures of doxorubicin, nemorubicin, and PNU-159682 are shown below.

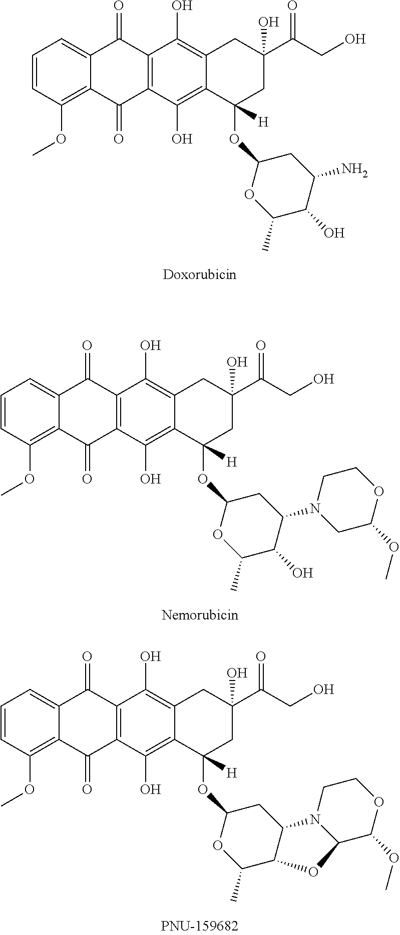 [SMILES: COc1cccc2c1C(=O)c1c(O)c3c(c(O)c1C2=O)C[C@@](O)(C(=O)CO)C[C@@H]3O[C@H]1C[C@H](N)[C@H](O)[C@H](C)O1 ; COc1cccc2c1C(=O)c1c(O)c3c(c(O)c1C2=O)C[C@@](O)(C(=O)CO)C[C@@H]3O[C@H]1C[C@H](N2CCO[C@H](OC)C2)[C@H](O)[C@H](C)O1 ; COc1cccc2c1C(=O)c1c(O)c3c(c(O)c1C2=O)C[C@@](O)(C(=O)CO)C[C@@H]3O[C@H]1C[C@H]2[C@H](O[C@@H]3[C@@H](OC)OCCN32)[C@H](C)O1]

The high cytotoxicity exhibited by PNU-159682 is related to its unique derivative structure, which can be used for various derivatives of anthracycline toxins. PNU-159682 is a metabolite of nemonoxacin, a derivative of whylin-based anthracycline toxins. Therefore, the methods disclosed in the literature are all based on modifying and derivatizing the structure of nemonoxacin to prepare PNU-159682. To obtain this unique derivative structure, traditional methods involve steps such as closing the morpholine ring, N-oxidation, and closing the oxazole ring, all starting from nemonoxacin or its intermediates. Sometimes, additional steps involving protection and deprotection groups are required, resulting in a lengthy route with relatively low yields at each step. The intermediates and products have poor stability, making it difficult to achieve large-scale synthesis.

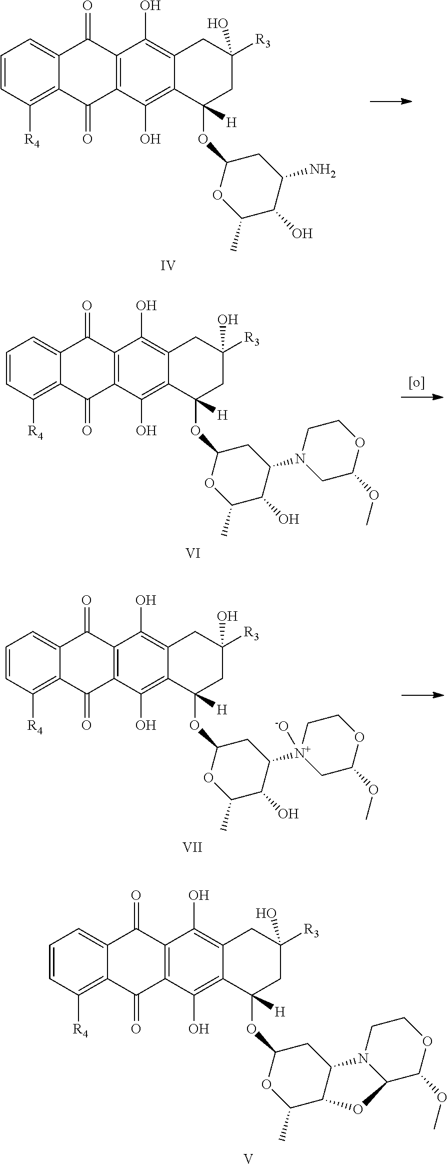

Taking the widely followed PNU-159682 as an example, the initial patent synthesis route (WO9802446A1, WO2012073217A1, WO2014177441, etc.) is shown as follows. Starting from amphotericin B (IVa), the compound is first cyclized to obtain namorubicin (VIa), namorubicin is then selectively oxidized to obtain compound (VIIa), and then cyclized to obtain the product PNU-159682. The entire route is relatively long, the reaction conditions are not easy to control, and the reaction scale is very small, only at the milligram level, making it difficult to scale up production.

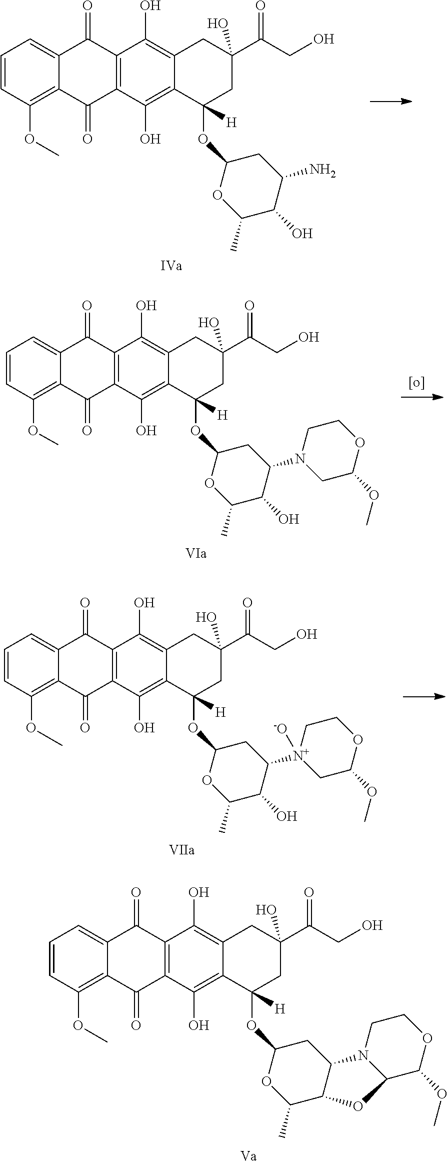

The subsequent patents (CN 103270043, CN 110776501, and WO2021031599A1, etc.) aim to improve the stability of raw materials and intermediates by using the expensive nemonapride (VIa) as a starting material and protecting the hydroxyl group, as shown below. Although the reaction steps have increased, the products are still obtained through selective oxidation and cyclization methods. While this approach has improved the stability of the reaction system to some extent, the additional steps of hydroxyl protection and deprotection have been added. The production process has not fundamentally improved, as issues such as unstable raw material sources, high prices, long production routes, and small production batches still exist.

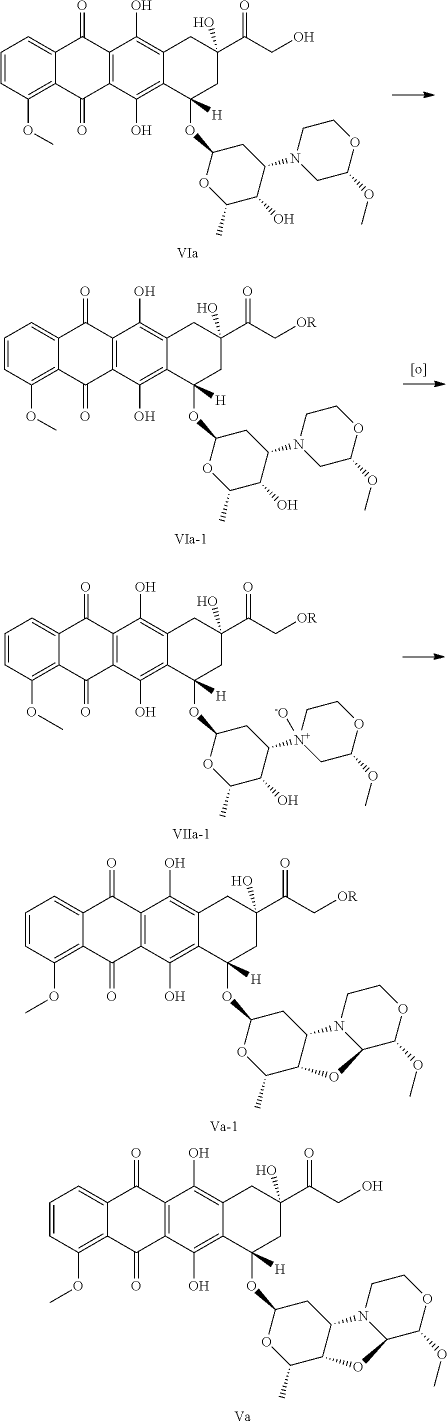

### SUMMARY OF INVENTION

This invention utilizes relatively inexpensive and readily available anthracene-like drugs or their derivatives (IV) as starting materials, cleverly designing an intermediate (III) that can be easily obtained through a one-step reaction to yield the corresponding pyran, benzoxazole, and benzothiazole compounds (V). Compound (III) is a newly reported compound, and its synthesis involves creative structural design, resulting in the efficient, stable, and convenient synthesis of the target compound (V).

To achieve the above-mentioned objectives, a intermediate compound is designed for the preparation of a class of anthracene-derived toxins, with the structural formula:

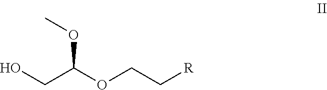

Wherein R is trifluoromethanesulfonyloxy, methanesulfonyloxy, benzenesulfonyloxy, p-toluenesulfonyloxy, p-fluorobenzenesulfonyloxy, o-nitrobenzenesulfonyloxy, p-nitrobenzenesulfonyloxy, chlorine (Cl), bromine (Br) or iodine (I).

The structural formula of the intermediate compound prepared from the above intermediate compound is:

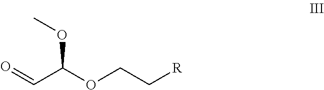

Wherein R is trifluoromethanesulfonyloxy, methanesulfonyloxy, phenylsulfonyloxy, p-tolylsulfonyloxy, p-fluorophenylsulfonyloxy, o-nitrophenylsulfonyloxy, p-nitrophenylsulfonyloxy, chlorine (Cl), bromine (Br) or iodine (I).

The synthesis method of intermediate compounds is as follows:

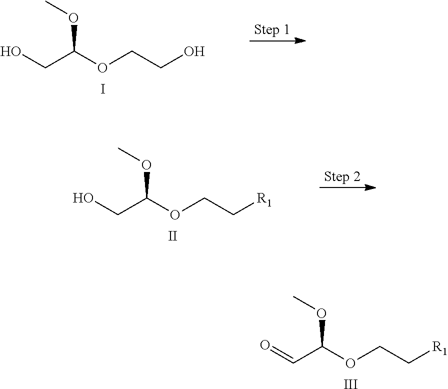

Step 1: Starting from (S)-2-(2-hydroxyethoxy)-2-methoxyethane-1-ol (I), synthesize the intermediate compound with the general structure II.

Step 2: Synthesize intermediate compounds with the structural formula II to obtain intermediate compounds with the structural formula III.

Step one is as follows:

1. Compound I is added to solvent A1, and reacts to form monosulfonic ester (II) under the action of reagents B1 and C1, with a reaction temperature ranging from −30° C. to 40° C.

Solvent A is selected from one or more of dichloromethane, 1,2-dichloroethane, chloroform, carbon tetrachloride, methyl tert-butyl ether, tetrahydrofuran, 2-methyltetrahydrofuran, isopropyl ether, diethyl ether, ethylene glycol dimethyl ether, acetonitrile, acetone, ethyl acetate, methyl acetate, isopropyl acetate, 1,4-dioxane, benzene, toluene, N,N-dimethylformamide, N,N-dimethylacetamide.

The reaction reagent B1 is selected from trifluoromethanesulfonyl anhydride, trifluoromethanesulfonyl chloride, 2-[N,N-bis(trifluoromethanesulfonyl)amino]pyridine, N,N-bis(trifluoromethanesulfonyl)-5-chloro-2-aminopyridine, N,N-bis(trifluoromethanesulfonyl)aniline, methanesulfonyl anhydride, methanesulfonyl chloride, 4-methylsulfonyl anhydride, 4-methylsulfonyl chloride, phenylsulfonyl sulfide, phenylsulfonyl chloride, 4-fluorophenylsulfonyl anhydride, 4-fluorophenylsulfonyl chloride, ortho-nitrophenylsulfonyl chloride, para-nitrophenylsulfonyl chloride, ortho-nitrophenylsulfonyl anhydride, or para-nitrophenylsulfonyl anhydride.

Reaction reagent C1 is selected from t-butylpotassium, t-butylsodium, butyllithium, diisopropylamine lithium, hexamethyldisilazane lithium, hexamethyldisilazane sodium, hexamethyldisilazane potassium, sodium hydride, triethylamine, N,N-diisopropylethylamine, pyridine, 2-methylpyridine, 2,6-dimethylpyridine, imidazole, 1-methylimidazole, 1-methylpiperidine, 4-methylmorpholine, tetramethylethylenediamine, or piperidine.

2. In solvent A2, compound I is added to react with reagents B2, C2, and D, resulting in the formation of mono-substituted halide (II) under reaction temperature ranging from −30 degrees to 40 degrees.

Solvent A2 is selected from one or more of dichloromethane, 1,2-dichloroethane, chloroform, carbon tetrachloride, methyl tert-butyl ether, tetrahydrofuran, 2-methyltetrahydrofuran, isopropyl ether, diethyl ether, ethylene glycol dimethyl ether, acetonitrile, acetone, ethyl acetate, methyl acetate, isopropyl acetate, dioxane, benzene, toluene, N,N-dimethylformamide, and N,N-dimethylacetamide.

The reaction reagent B2 is selected from carbon tetrachloride, hexachloroacetone, N-chlorosuccinimide, liquid bromine, carbon tetrabromide, trichlorobromomethane, N-bromosuccinimide, iodomethane, iodoethane, diiodomethane, iodoform, N-iodosuccinimide or iodine.

The reaction reagent C2 is selected from triphenylphosphine or triphenylphosphate in organic chemistry.

The reaction reagent D is selected from triethylamine, N,N-diisopropylethylamine, pyridine, 2-methylpyridine, 2,6-dimethylpyridine, imidazole, 1-methylimidazole, 1-methylpiperidine, 4-methylmorpholine, tetramethylethylenediamine or piperidine.

3. In solvent A3, compound I is added to react with reagents B3 and C3 to generate mono-substituted halide (II) at a reaction temperature of −30 to 40 degrees.

Solvent A3 is selected from one or more of dichloromethane, 1,2-dichloroethane, chloroform, carbon tetrachloride, methyl tert-butyl ether, tetrahydrofuran, 2-methyltetrahydrofuran, isopropyl ether, diethyl ether, ethylene glycol dimethyl ether, acetonitrile, acetone, ethyl acetate, methyl acetate, isopropyl acetate, 1,4-dioxane, benzene, toluene, N,N-dimethylformamide, and N,N-dimethylacetamide in organic professional English.

The reaction reagent B3 is selected from sulfuryl chloride, phosphorus trichloride, phosphorus oxychloride, phosphorus pentachloride, phosphorus tribromide, phosphorus oxybromide, or phosphorus pentabromide.

The reaction reagent C3 is selected from triethylamine, N,N-diisopropylethylamine, pyridine, 2-methylpyridine, 2,6-dimethylpyridine, imidazole, 1-methylimidazole, 1-methylpiperidine, 4-methylmorpholine, tetramethylethylenediamine, or piperidine.

Step two is as follows:

In solvent E, intermediate compound II and reaction reagent F are added to generate intermediate compound III. Reaction reagent F can be a single reagent or a combination of multiple reagents, with reaction temperature ranging from −70 degrees to 40 degrees.

Solvent E is selected from one or more of dichloromethane, 1,2-dichloroethane, chloroform, carbon tetrachloride, methyl tert-butyl ether, tetrahydrofuran, 2-methyltetrahydrofuran, isopropyl ether, diethyl ether, ethylene glycol dimethyl ether, acetonitrile, acetone, ethyl acetate, methyl acetate, isopropyl acetate, 1,4-dioxane, benzene, toluene, N,N-dimethylformamide, N,N-dimethylacetamide.

The reagent F for the mentioned reaction is selected from 2-iodobenzoic acid, Jones reagent, Collins reagent, chromium(III) chloride pyridine complex, chromium(VI) chloride pyridine complex, Dess-Martin oxidant, meta-chloroperoxybenzoic acid, acyl chloride/dimethyl sulfoxide/triethylamine combination, trifluoromethanesulfonyl anhydride/dimethyl sulfoxide/N,N-diisopropylethylamine combination, sodium nitrite/acetic anhydride combination, or hexamethylphosphoramide/dimethyl sulfoxide/dichloroacetic acid combination.

There is another intermediate compound used for the preparation of a class of anthracene-derived toxins, with the structural formula:

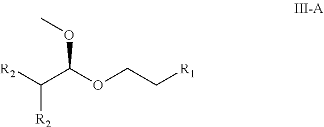

R1 can be trifluoromethanesulfonyloxy, methanesulfonyloxy, benzenesulfonyloxy, para-toluenesulfonyloxy, para-fluorobenzenesulfonyloxy, ortho-nitrobenzenesulfonyloxy, para-nitrobenzenesulfonyloxy, chlorine (Cl), bromine (Br) or iodine (I).

R2 can be an aldehyde, a diester, or a dihalide. When R2 is an aldehyde, it can be methoxy, ethoxy, 1-propoxy, 1-butoxy, 1-pentoxy, ethylenedioxy, 2,3-butanedioxy, 1,2-diphenylethylenedioxy, 1,2-propylenedioxy, 1,3-propylenedioxy, 2-methyl-1,3-propylenedioxy, 2-ethyl-1,3-propylenedioxy, 2-n-propyl-1,3-propylenedioxy, 2-ethyl-2-methyl-1,3-propylenedioxy, 2-isopropylpropane-1,3-dioxy, 2-tert-butylpropane-1,3-dioxy, 2-methoxy-1,3-propylenedioxy, 2,2-dimethyl-1,3-propylenedioxy, 2,2-ethyl-1,3-propylenedioxy, 1,1-cyclopentanedimethoxy, 1,1-cyclobutanedimethoxy, 1,1-cyclopentanedimethoxy, 1,1-cyclohexanedimethoxy, 1,3-butanedioxy, 2-methyl-1,3-butanedioxy, 1,3-pentanedioxy, 2,4-pentanedioxy; When R2 is a diester, it can be acetoxy, propionyloxy, butyryloxy, valeroyloxy, pentanoyloxy, trifluoroacetoxy, trifluoromethanesulfonyloxy, methanesulfonyloxy, benzenesulfonyloxy, p-toluenesulfonyloxy, p-fluorobenzenesulfonyloxy, o-nitrobenzenesulfonyloxy, p-nitrobenzenesulfonyloxy; When R2 is a dihalide, it can be chlorine (Cl), bromine (Br), or iodine (I).

The synthesis methods of the above-mentioned intermediate compounds are as follows:

In solvent G, compound III and reagent H are added to generate intermediate compound III-A. Reagent H can be a single reagent or a combination of multiple reagents, with reaction temperature ranging from −70° C. to 70° C.

Solvent G is selected from one or more of dichloromethane, 1,2-dichloroethane, chloroform, carbon tetrachloride, methyl tert-butyl ether, tetrahydrofuran, 2-methyltetrahydrofuran, isopropyl ether, ether, ethylene glycol dimethyl ether, methanol, ethanol, 1-propanol, acetonitrile, acetone, butanone, ethyl acetate, methyl acetate, isopropyl acetate, 1,4-dioxane, benzene, toluene, N,N-dimethylformamide, N,N-dimethylacetamide, or carbon disulfide; preferably dichloromethane, chloroform, methanol, 2-methyltetrahydrofuran, and carbon disulfide.

1. When the aldehyde group of compound II is converted to an acetal, the reagent H is selected from original methyl formate, original ethyl formate, 1-butanol, 1-pentanol, ethylene glycol, 1,2-propanediol, 1,3-propanediol, 2-methyl-1,3-propanediol, 2-ethyl-1,3-propanediol, 2-n-propyl-1,3-propanediol, 2-ethyl-2-methyl-1,3-propanediol, 2-isopropylpropane-1,3-diol, 2-tert-butylpropane-1,3-diol, 2-methoxy-1,3-propanediol, 2,2-dimethyl-1,3-propanediol, 2,2-ethyl-1,3-propanediol, 1,1-cyclopropanedimethanol, 1,1-di(hydroxymethyl)cyclobutane, 1,1-di(hydroxymethyl)cyclopentane, 1,1-di(hydroxymethyl)cyclohexane, 1,3-butanediol, 2-methyl-1,3-butanediol, 1,3-pentanediol, 2,4-pentanediol, pinacol, hydrogenated benzoin, resorcinol, hydrochloric acid, hydrobromic acid, hydroiodic acid, sulfuric acid, methylsulfonic acid, ethylsulfonic acid, propylsulfonic acid, butylsulfonic acid, camphorsulfonic acid, p-toluenesulfonic acid, trifluoromethanesulfonic acid, perfluorobutanesulfonic acid, perfluorohexanesulfonic acid, perfluoroheptanesulfonic acid, perfluorooctanesulfonic acid, and one or more of the corresponding metal salts, pyridine salts, imidazole salts, N-methylimidazole salts; when the acetal is specialized to a diester, the reagent H is selected from acetic anhydride, propionic anhydride, butyric anhydride, valeric anhydride, pivalic anhydride, trifluoroacetic anhydride, trifluoromethanesulfonic anhydride, methanesulfonic anhydride, benzenesulfonic anhydride, p-toluenesulfonic anhydride, p-fluorobenzenesulfonic anhydride, o-nitrobenzenesulfonic anhydride, p-nitrobenzenesulfonic anhydride, triethylamine, N,N-diisopropylethylamine, pyridine, 2-methylpyridine, 2,6-dimethylpyridine, imidazole, 1-methylimidazole, 1-methylpiperidine, 4-methylmorpholine, tetramethyl ethylenediamine, one or more of p-dimethylaminopyridine; when the diester is specialized to a dihalide, the reagent H is selected from magnesium chloride, magnesium bromide, magnesium iodide, zinc chloride, zinc bromide, zinc iodide, lithium chloride, lithium bromide, lithium iodide, sodium chloride, sodium bromide, sodium iodide, potassium chloride, potassium bromide, potassium iodide, tetrabutylammonium chloride, tetrabutylammonium bromide, tetrabutylammonium iodide, tetraethylammonium bromide, tetraethylammonium iodide, benzyltriethylammonium bromide, benzyltriethylammonium iodide.

2. In solvent E, compound III-1 and reagent F are added to generate intermediate compound III-A. Reagent F can be a single reagent or a combination of multiple reagents, and the reaction temperature ranges from −70° C. to 60° C.

Solvent E is selected from one or more of dichloromethane, 1,2-dichloroethane, chloroform, carbon tetrachloride, methyl tert-butyl ether, tetrahydrofuran, 2-methyltetrahydrofuran, isopropyl ether, diethyl ether, ethylene glycol dimethyl ether, acetonitrile, acetone, butanone, ethyl acetate, methyl acetate, isopropyl acetate, 1,4-dioxane, benzene, toluene, N,N-dimethylformamide, N,N-dimethylacetamide.

The reaction reagent F mentioned above is selected from 2-iodobenzoic acid, Jones reagent, Collins reagent, pyridinium chlorochromate, pyridinium chlorochromate, Dess-Martin oxidant, m-chloroperoxybenzoic acid, acyl chloride/dimethyl sulfoxide/triethylamine combination, trifluoromethanesulfonyl anhydride/dimethyl sulfoxide/N,N-diisopropylethylamine combination, sodium nitrite/acetic anhydride combination, dicyclohexylcarbodiimide/dimethyl sulfoxide/dichloroacetic acid combination.

The preparation method of a class of anthracene ring-like toxin derivatives involves the preparation of intermediate compounds.

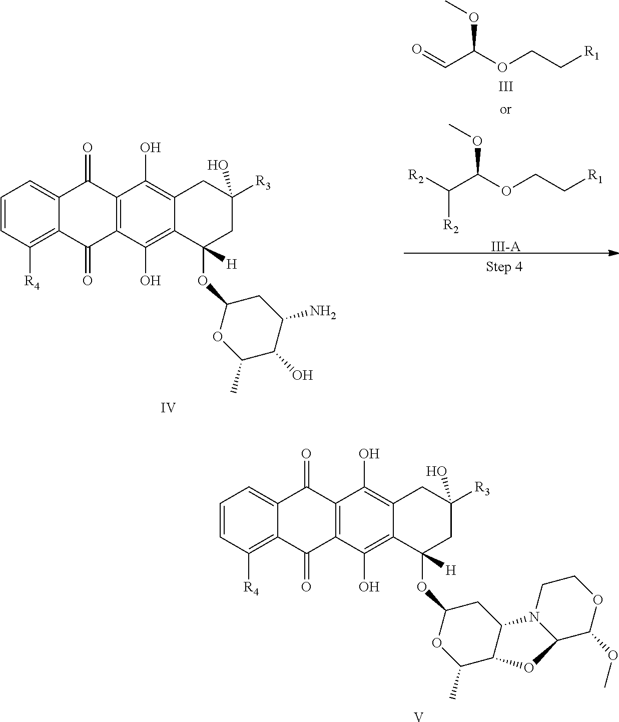

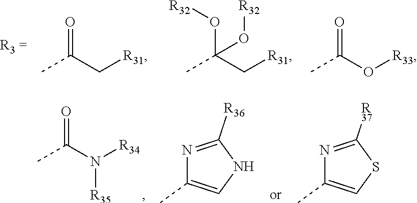

In solvent I, intermediate compound III or III-A, compound IV, and reagent J are added to react and produce compound V at a reaction temperature ranging from −30 degrees to 80 degrees.

Solvent I is selected from one or more of dichloromethane, 1,2-dichloroethane, chloroform, carbon tetrachloride, methyl tert-butyl ether, tetrahydrofuran, 2-methyltetrahydrofuran, isopropyl ether, diethyl ether, ethylene glycol dimethyl ether, acetonitrile, acetone, ethyl acetate, methyl acetate, isopropyl acetate, 1,4-dioxane, benzene, toluene, N,N-dimethylformamide, N,N-dimethylacetamide; and reagent J is selected from one or more of sodium bicarbonate, sodium carbonate, potassium bicarbonate, potassium carbonate, cesium carbonate, potassium phosphate, sodium phosphate, potassium dihydrogen phosphate, sodium dihydrogen phosphate, sodium hydroxide, potassium hydroxide, triethylamine, N,N-diisopropylethylamine, pyridine, 2-methylpyridine, 2,6-dimethylpyridine, imidazole, 1-methylimidazole, 1-methylpiperidine, 4-methylmorpholine, tetramethyl ethylenediamine or piperidine.

The structure of compound V is as follows:

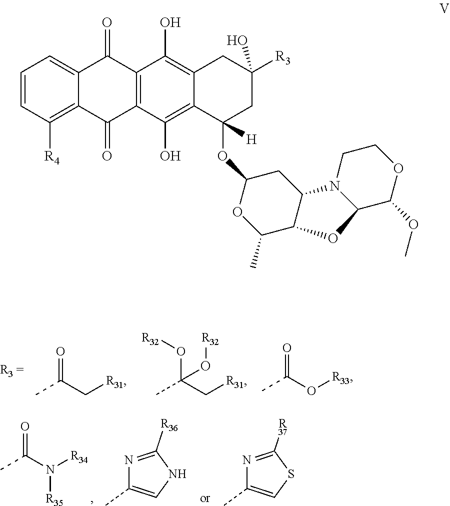

R2 can be hydrogen (H), fluorine (F), chlorine (Cl), bromine (Br), iodine (I), hydroxyl (OH), amino (NH2), hydrazino (NHNH2), thiol (SH), trifluoromethanesulfonyloxy, methanesulfonyloxy, benzenesulfonyloxy, para-toluenesulfonyloxy, para-fluorobenzenesulfonyloxy, ortho-nitrobenzenesulfonyloxy, para-nitrobenzenesulfonyloxy, acetyloxy, trifluoroacetyloxy, benzoyloxy, tert-butoxy, benzyl-oxy, para-methoxybenzyl-oxy, 3,4-dimethoxybenzyl-oxy, triphenylmethoxy, 2-tetrahydropyranyloxy, methoxymethoxy, 2-ethoxyethoxy, 2-(trimethylsilyl)ethoxy-methoxy, allyloxy, phenoxy-carbonyloxy, 4-nitrophenoxy-carbonyloxy, pentafluorophenoxy-carbonyloxy, trimethylsiloxy, tert-butyldimethylsiloxy, tert-butyldiphenylsiloxy, diethylisopropylsiloxy, triisopropylsiloxy, triphenylsiloxy, trifluoromethanesulfonylamino, methanesulfonylamino, benzenesulfonylamino, para-toluenesulfonylamino, para-fluorobenzenesulfonylamino, ortho-nitrobenzenesulfonylamino, para-nitrobenzenesulfonylamino, 2-(trimethylsilyl)ethylsulfonyl, acetylamino, trifluoroacetylamino, benzoylamino, tert-butoxycarbonylamino, ortho-benzoylhydrazino, benzylamino, para-methoxybenzylamino, 3,4-dimethoxybenzylamino, allylamino, triphenylamino, methoxycarbonylamino, ethoxycarbonylamino, benzylcarbonylamino, tert-butoxycarbonylamino, fluorenylmethoxycarbonylamino, allyloxycarbonylamino, trimethylsilyl-ethoxycarbonylamino, trichloroethoxycarbonylamino, various derivatives of alkoxy and alkylamine linkages, etc.

Among them, R31 is hydrogen H, fluorine F, chlorine Cl, bromine Br, iodine I, hydroxyl OH, amino NH2, hydrazine NHNH2, thiol SH, trifluoromethanesulfonyloxy, methanesulfonyloxy, benzenesulfonyloxy, para-toluenesulfonyloxy, para-fluorobenzenesulfonyloxy, ortho-nitrobenzenesulfonyloxy, para-nitrobenzenesulfonyloxy, acetyloxy, trifluoroacetyloxy, benzoyloxy, pivaloyloxy, benzyl oxy, para-methoxybenzyl oxy, 3,4-dimethoxybenzyl oxy, triphenylmethoxy, 2-tetrahydropyranyloxy, methoxymethoxy, 2-ethoxyethoxy, 2-(trimethylsilyl)ethoxymethoxy, allyloxy, phenoxy carbonyloxy, 4-nitrophenoxy carbonyloxy, pentafluorophenoxy carbonyloxy, trimethoxysilane, tert-butyldimethylsiloxy, tert-butyldiphenylsiloxy, diethylisopropylsiloxy, triisopropylsiloxy, triphenylsiloxy, trifluoromethanesulfonylamino, methanesulfonylamino, benzenesulfonylamino, para-toluenesulfonylamino, para-fluorobenzenesulfonylamino, ortho-nitrobenzenesulfonylamino, para-nitrobenzenesulfonylamino, 2-(trimethylsilyl)ethanesulfonyl, acetylamino, trifluoroacetylamino, benzoylamino, pivaloylamino, ortho-phenylenediamine, benzylamino, para-methoxybenzylamino, 3,4-dimethoxybenzylamino, allylamino, triphenylmethylamino, methoxycarbonylamino, ethoxycarbonylamino, benzylcarbonylamino, tert-butyloxycarbonylamino, fluorenylmethoxycarbonylamino, allyloxycarbonylamino, trimethylsiloxyethoxycarbonylamino, trichloroethoxycarbonylamino or various derivatives of alkoxyl and alkylamine connections.

Among them, R32 is a ketone protective group derived from methanol, ethanol, 1-propanol, 1-butanol, 1-pentanol, ethylene glycol, 1,2-propanediol, 1,3-propanediol, 2-methyl-1,3-propanediol, 2-ethyl-1,3-propanediol, 2-n-propyl-1,3-propanediol, 2-ethyl-2-methyl-1,3-propanediol, 2-isopropylpropane-1,3-diol, 2-tert-butylpropane-1,3-diol, 2-methoxy-1,3-propanediol, 2,2-dimethyl-1,3-propanediol, 2,2-ethyl-1,3-propanediol, 1,1-cyclopropanedimethanol, 1,1-di(hydroxymethyl)cyclobutane, 1,1-di(hydroxymethyl)cyclopentane, 1,1-di(hydroxymethyl)cyclohexane, 1,3-butanediol, 2-methyl-1,3-butanediol, 1,3-pentanediol, or 2,4-pentanediol.

R33, R34, R35, R36, and R37 are alkyl or aryl groups, such as hydrogen (H), methyl, ethyl, propyl, isopropyl, tert-butyl, benzyl, allyl, para-methoxybenzyl, 3,4-dimethoxybenzyl, triphenylmethyl, or various derivatives thereof, connected by oxygen or amine linkages.

The advantages of this invention compared to existing technologies are as follows: the preparation method of this invention uses relatively inexpensive and easily accessible compound (IV) as the starting material, and cleverly designed intermediate (III), which can be obtained through a one-step reaction to yield corresponding pyran, oxazole, and oxadiazole compounds (V). The steps of this invention are concise, the process is stable and reliable, easy to scale up production, effectively increasing the batch yield and production efficiency. This invention creatively uses commercially available anthracene drugs or their analogues or derivatives as starting materials, effectively solving the problems of restricted materials and high costs. These commercially available materials are cheap, with abundant market supply, greatly reducing the raw material costs for producing compounds (V) and ensuring a stable supply of raw materials for large-scale production. The simplification of the synthetic steps greatly saves labor costs and time costs, effectively reducing the complexity of product impurity spectra, controlling the types and contents of impurities, significantly increasing the product yield and quality.

### DETAILED DESCRIPTION OF INVENTION

The following further explains the present invention in conjunction with the accompanying diagram. The structure and principle of the present invention are very clear to professionals in this field. It should be understood that the specific embodiments described herein are only used to illustrate the present invention and are not intended to limit the present invention.

The preparation method of this invention starts with the relatively inexpensive and readily available anthracene class drugs or their derivatives IV as the starting materials. Intermediate III and III-A are cleverly designed, and through a one-step reaction, the corresponding pyran and oxazole compounds V can be obtained. Compounds III and III-A are newly reported compounds, and we used creative structural design in the synthesis to efficiently, conveniently, and stably obtain the target compound V.

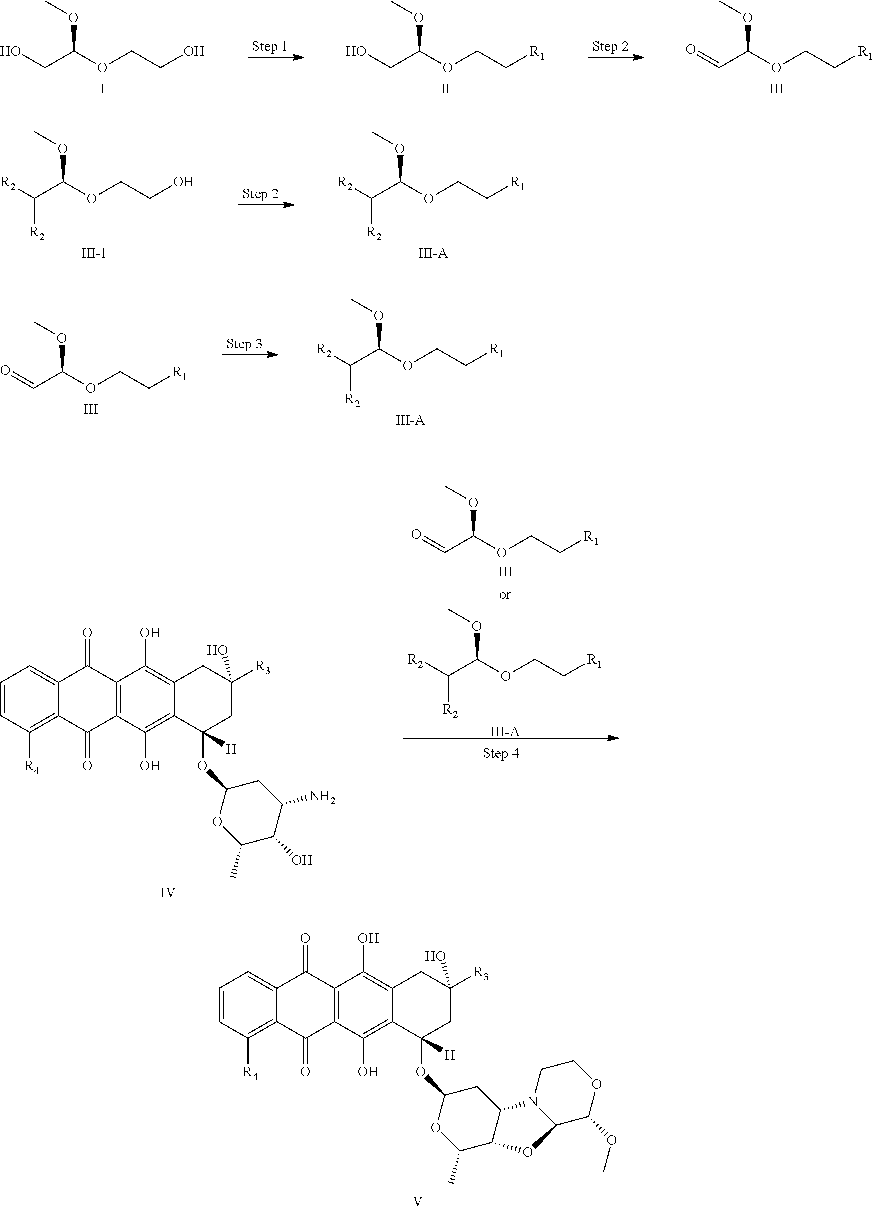

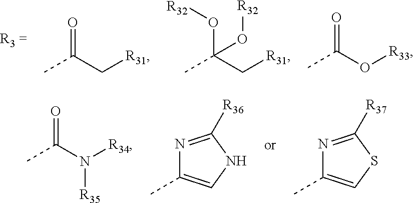

### Step One, Starting from (S)-2-(2-hydroxyethoxy)-2-methoxyethane-1-ol (compound I), Compound II with the General Structure can be Obtained Through Various Synthetic Methods, where the R Group of Compound II is Sulfonyloxy or Halogen, Etc.

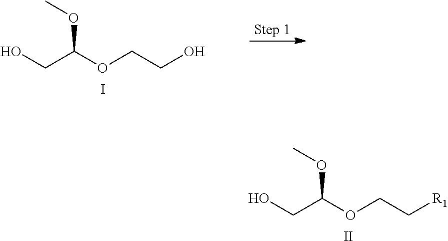

Method 1: Compound I is added to solvent A1, and reacts with reagents B1 and C1 to generate sulfonyl ester II, with a reaction temperature range of −30 degrees to 70 degrees, preferably at 30 to 40 degrees. Solvent A can be selected from dichloromethane, 1,2-dichloroethane, chloroform, carbon tetrachloride, methyl tert-butyl ether, tetrahydrofuran, 2-methyltetrahydrofuran, isopropyl ether, ethyl ether, ethylene glycol dimethyl ether, acetonitrile, acetone, ethyl acetate, methyl acetate, isopropyl acetate, 1,4-dioxane, benzene, toluene, N,N-dimethylformamide, and N,N-dimethylacetamide, with dichloromethane, methyl tert-butyl ether, and tetrahydrofuran being preferred.

Reagent B1 is selected from trifluoromethanesulfonyl anhydride, trifluoromethanesulfonyl chloride, 2-[N,N-bis(trifluoromethanesulfonyl)amino]pyridine, N,N-bis(trifluoromethanesulfonyl)-5-chloro-2-aminopyridine, N,N-bis(trifluoromethanesulfonyl)aniline, methanesulfonyl anhydride, methanesulfonyl chloride, 4-toluenesulfonyl anhydride, 4-toluenesulfonyl chloride, phenylsulfonyl sulfide, phenylsulfonyl chloride, 4-fluorobenzenesulfonyl anhydride, 4-fluorobenzenesulfonyl chloride, ortho-nitrobenzenesulfonyl chloride, para-nitrobenzenesulfonyl chloride, ortho-nitrobenzenesulfonyl anhydride, para-nitrobenzenesulfonyl anhydride, etc.

Reaction reagent C1 is selected from tert-butyl potassium, tert-butyl sodium, butyl lithium, diisopropylamine lithium, hexamethyldisilazane lithium, hexamethyldisilazane sodium, hexamethyldisilazane potassium, sodium hydride, triethylamine, N,N-diisopropylethylamine, pyridine, 2-methylpyridine, 2,6-dimethylpyridine, imidazole, 1-methylimidazole, 1-methylpiperidine, 4-methylmorpholine, tetramethylethylenediamine, piperidine, and other compounds.

Method 2: Compound I is added to solvent A2, and reacts to generate monosubstituted halide II under the action of reagents B2, C2, and D, with reaction temperature ranging from 0° C. to 80° C., preferably at 30° C. to 40° C. Solvent A2 can be selected from dichloromethane, 1,2-dichloroethane, chloroform, carbon tetrachloride, methyl tert-butyl ether, tetrahydrofuran, 2-methyltetrahydrofuran, isopropyl ether, ether, ethylene glycol dimethyl ether, acetonitrile, acetone, ethyl acetate, methyl acetate, isopropyl acetate, 1,4-dioxane, benzene, toluene, N,N-dimethylformamide, N,N-dimethylacetamide, or a combination thereof; with dichloromethane and tetrahydrofuran being preferred. Reagent B2 can be selected from carbon tetrachloride, hexachloroacetone, N-chlorosuccinimide, liquid bromine, carbon tetrabromide, chlorobromomethane, N-bromosuccinimide, iodomethane, iodoethane, diiodomethane, iodoform, N-iodosuccinimide, iodine, etc. Reagent C2 can be selected from triphenylphosphine or triphenylphosphite. Reagent D can be selected from triethylamine, N,N-diisopropylethylamine, pyridine, 2-methylpyridine, 2,6-dimethylpyridine, imidazole, 1-methylimidazole, 1-methylpiperidine, 4-methylmorpholine, tetramethylethylenediamine, piperidine, etc.

Method 3: Compound I is added to solvent A3, and reacts with reagents B3 and C3 to generate monosubstituted halide II. The reaction temperature ranges from 0° C. to 80° C., with preferred temperature between 30° C. to 40° C. Solvent A3 can be selected from dichloromethane, 1,2-dichloroethane, chloroform, carbon tetrachloride, methyl tert-butyl ether, tetrahydrofuran, 2-methyltetrahydrofuran, isopropyl ether, ether, ethylene glycol dimethyl ether, acetonitrile, acetone, ethyl acetate, methyl acetate, isopropyl acetate, 1,4-dioxane, benzene, toluene, N,N-dimethylformamide, N,N-dimethylacetamide, or a combination of these; with preferred options being dichloromethane, benzene, and methyl tert-butyl ether. Reagents B3 can be selected from sulfonyl chloride, phosphorus trichloride, phosphorus oxychloride, phosphorus pentachloride, phosphorus tribromide, phosphorus oxybromide, or phosphorus pentabromide. Reagents C3 can be selected from triethylamine, N,N-diisopropylethylamine, pyridine, 2-methylpyridine, 2,6-dimethylpyridine, imidazole, 1-methylimidazole, 1-methylpiperidine, 4-methylmorpholine, tetramethyl ethylenediamine, piperidine, or a combination of these.

This patent also provides intermediate compounds with the following structural formula.

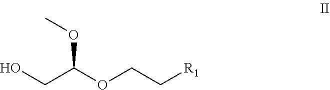

R1 can be trifluoromethanesulfonyloxy, methanesulfonyloxy, phenylsulfonyloxy, para-toluenesulfonyloxy, para-fluorophenylsulfonyloxy, ortho-nitrophenylsulfonyloxy, para-nitrophenylsulfonyloxy, chlorine (Cl), bromine (Br) or iodine (I), etc.

Compound II is a newly synthesized compound that has never been reported before, utilizing a creative structural design in its synthesis.

Step two is the method for synthesizing intermediate compounds III and III-A. Compound II or III-1 is added to solvent E along with reagent F to generate intermediate compounds III or III-A. Reagent F can be a single reagent or a combination of multiple reagents, with a reaction temperature ranging from −70° C. to 60° C.

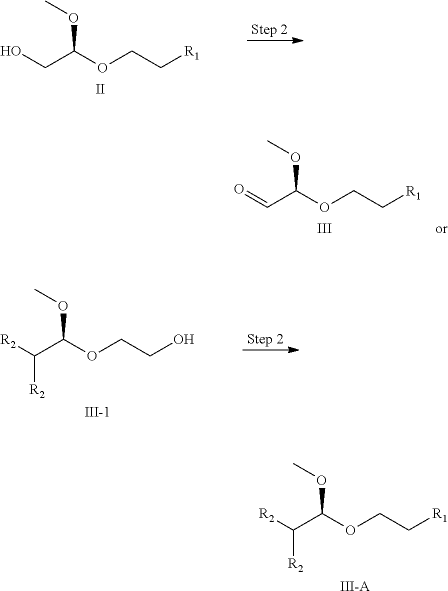

Among them, R1 is trifluoromethanesulfonyloxy, methanesulfonyloxy, benzenesulfonyloxy, p-toluenesulfonyloxy, p-fluorobenzenesulfonyloxy, o-nitrobenzenesulfonyloxy, p-nitrobenzenesulfonyloxy, chlorine (Cl), bromine (Br), or iodine (I), etc.

Solvent E is selected from one or more of dichloromethane, 1,2-dichloroethane, chloroform, carbon tetrachloride, methyl tert-butyl ether, tetrahydrofuran, 2-methyltetrahydrofuran, isopropyl ether, diethyl ether, ethylene glycol dimethyl ether, acetonitrile, acetone, butanone, ethyl acetate, methyl acetate, isopropyl acetate, 1,4-dioxane, benzene, toluene, N,N-dimethylformamide, N,N-dimethylacetamide; preferably dichloromethane, chloroform, and 2-methyltetrahydrofuran.

Reaction reagent F is selected from 2-iodobenzoic acid, Jones reagent, Collins reagent, pyridinium chlorochromate, pyridinium chlorochromate, Dess-Martin oxidant, m-chloroperbenzoic acid, acetyl chloride/dimethyl sulfoxide/triethylamine combination, trifluoromethanesulfonyl anhydride/dimethyl sulfoxide/N,N-diisopropylethylamine combination, sodium nitrite/acetic anhydride combination, dicyclohexylcarbodiimide/dimethyl sulfoxide/dichloroacetic acid combination; preferred reagents include trifluoromethanesulfonyl anhydride/dimethyl sulfoxide/N,N-diisopropylethylamine combination, sodium nitrite/acetic anhydride combination, and Dess-Martin oxidant.

Where R2 is an aldehyde, diester, or dihalogenated compound, R2 can be methoxy, ethoxy, 1-propoxy, 1-butoxy, 1-pentoxy, ethylenedioxy, 2,3-butanedioxy, 1,2-diphenylethylenedioxy, ortho-benzyloxy, 1,2-propylenedioxy, 1,3-propylenedioxy, 2-methyl-1,3-propylenedioxy, 2-ethyl-1,3-propylenedioxy, 2-n-propyl-1,3-propylenedioxy, 2-ethyl-2-methyl-1,3-propylenedioxy, 2-isopropylpropane-1,3-diyoxy, 2-tert-butylpropane-1,3-diyoxy, 2-methoxy-1,3-propylenedioxy, 2,2-dimethyl-1,3-propylenedioxy, 2,2-ethyl-1,3-propylenedioxy, 1,1-cyclopropane dimethoxy, 1,1-cyclobutane dimethoxy, 1,1-cyclopentane dimethoxy, 1,1-cyclohexane dimethoxy, 1,3-butanedioxy, 2-methyl-1,3-butanedioxy, 1,3-pentanedioxy, 2,4-pentanedioxy, etc. When R2 is a diester, it can be acetoxy, propionyloxy, butyryloxy, valeroyloxy, pentanoyloxy, trifluoroacetoxy, trifluoromethanesulfonyloxy, methanesulfonyloxy, benzenesulfonyloxy, para-toluenesulfonyloxy, para-fluorobenzenesulfonyloxy, ortho-nitrobenzenesulfonyloxy, para-nitrobenzenesulfonyloxy, etc. When R2 is a dihalogenated compound, it can be chlorine (Cl), bromine (Br), or iodine (I), etc.

This patent also provides intermediate compounds III and III-A with the following structural formulas.

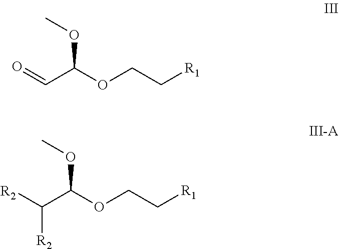

R1 is trifluoromethanesulfonyloxy, methanesulfonyloxy, benzenesulfonyloxy, p-toluenesulfonyloxy, p-fluorobenzenesulfonyloxy, o-nitrobenzenesulfonyloxy, p-nitrobenzenesulfonyloxy, chlorine (Cl), bromine (Br) or iodine (I), etc. R2 is an aldehyde or a diester or a dihalogen compound. When R2 is an aldehyde, it can be methoxy, ethoxy, 1-propoxy, 1-butoxy, 1-pentoxy, ethylenedioxy, 2,3-butanedioxy, 1,2-diphenylethylenedioxy, 1,2-propanedioxy, 1,3-propanedioxy, 2-methyl-1,3-propanedioxy, 2-ethyl-1,3-propanedioxy, 2-n-propyl-1,3-propanedioxy, 2-ethyl-2-methyl-1,3-propanedioxy, 2-isopropylpropane-1,3-dioxy, 2-tert-butylpropane-1,3-dioxy, 2-methoxy-1,3-propanedioxy, 2,2-dimethyl-1,3-propanedioxy, 2,2-ethyl-1,3-propanedioxy, 1,1-cyclopropyl dimethoxy, 1,1-cyclobutyl dimethoxy, 1,1-cyclopentyl dimethoxy, 1,1-cyclohexyl dimethoxy, 1,3-butanedioxy, 2-methyl-1,3-butanedioxy, 1,3-pentanedioxy, 2,4-pentanedioxy, etc. When R2 is a diester, it can be acetyloxy, propionyloxy, butyryloxy, valeroyloxy, pentanoyloxy, trifluoroethanoyloxy, trifluoromethanesulfonyloxy, methanesulfonyloxy, benzenesulfonyloxy, p-toluenesulfonyloxy, p-fluorobenzenesulfonyloxy, o-nitrobenzenesulfonyloxy, p-nitrobenzenesulfonyloxy, etc. When R2 is a dihalogen compound, it can be chlorine (Cl), bromine (Br) or iodine (I), etc.

Compounds III and III-A are previously unreported new compounds, for which we utilized creative structural design in the synthesis. The typical structural representations of Compounds III and III-A are as follows:

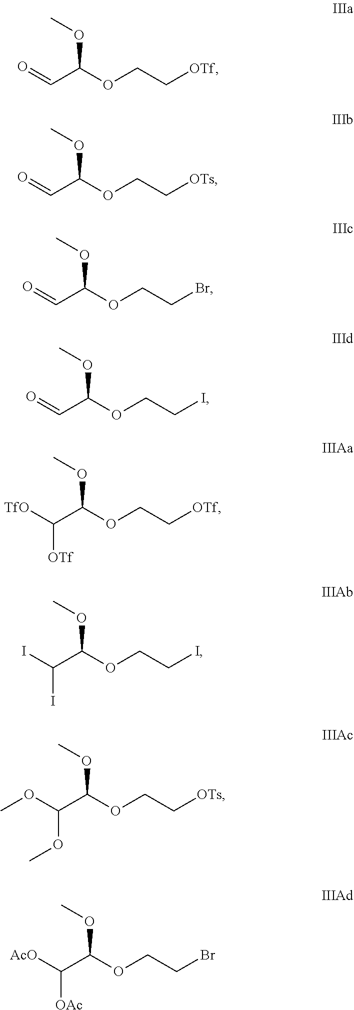

Step three is a supplementary method for the synthesis of intermediate compound III-A. In solvent G, compound III and reaction reagent H are added to react and generate intermediate compound III-A. Reaction reagent H can be a single reagent or a combination of multiple reagents, and the reaction temperature ranges from −70 to 70 degrees.

Among them, R1 is trifluoromethanesulfonyloxy, methanesulfonyloxy, benzenesulfonyloxy, para-toluenesulfonyloxy, para-fluorobenzenesulfonyloxy, ortho-nitrobenzenesulfonyloxy, para-nitrobenzenesulfonyloxy, chlorine (Cl), bromine (Br), or iodine (I), etc.

Solvent G is selected from one or more of dichloromethane, 1,2-dichloroethane, chloroform, carbon tetrachloride, methyl tert-butyl ether, tetrahydrofuran, 2-methyltetrahydrofuran, isopropyl ether, diethyl ether, ethylene glycol dimethyl ether, methanol, ethanol, 1-propanol, acetonitrile, acetone, butanone, ethyl acetate, methyl acetate, isopropyl acetate, 1,4-dioxane, benzene, toluene, N,N-dimethylformamide, N,N-dimethylacetamide, or carbon disulfide; preferably dichloromethane, chloroform, methanol, 2-methyltetrahydrofuran, and carbon disulfide.

When the aldehyde group of compound II is converted to an acetal, the reaction reagent H is selected from original trimethyl orthoformate, original triethyl orthoformate, 1-butanol, 1-pentanol, ethylene glycol, 1,2-propanediol, 1,3-propanediol, 2-methyl-1,3-propanediol, 2-ethyl-1,3-propanediol, 2-n-propyl-1,3-propanediol, 2-ethyl-2-methyl-1,3-propanediol, 2-isopropyl-1,3-propanediol, 2-tert-butyl-1,3-propanediol, 2-methoxy-1,3-propanediol, 2,2-dimethyl-1,3-propanediol, 2,2-ethyl-1,3-propanediol, 1,1-cyclohexanedimethanol, 1,1-di(hydroxymethyl)cyclobutane, 1,1-di(hydroxymethyl)cyclopentane, 1,1-di(hydroxymethyl)cyclohexane, 1,3-butanediol, 2-methyl-1,3-butanediol, 1,3-pentanediol, 2,4-pentanediol, pentaerythritol, hydrogenated hydroquinone, resorcinol, hydrogen chloride, hydrogen bromide, hydrogen iodide, sulfuric acid, methylsulfonic acid, ethylsulfonic acid, propylsulfonic acid, butylsulfonic acid, camphorsulfonic acid, p-toluenesulfonic acid, trifluoromethanesulfonic acid, perfluorobutanesulfonic acid, perfluorohexanesulfonic acid, perfluoroheptanesulfonic acid, perfluorooctanesulfonic acid, and one or more of the corresponding metal salts, pyridine salts, imidazole salts, or N-methylimidazole salts. When the acetal is specialized into a diester, the reaction reagent H is selected from acetic anhydride, propionic anhydride, butyric anhydride, valeric anhydride, pivalic anhydride, trifluoroacetic anhydride, trifluoromethanesulfonic anhydride, methanesulfonic anhydride, benzenesulfonic anhydride, p-toluenesulfonic anhydride, p-fluorobenzenesulfonic anhydride, o-nitrobenzenesulfonic anhydride, p-nitrobenzenesulfonic anhydride, triethylamine, N,N-diisopropylethylamine, pyridine, 2-methylpyridine, 2,6-dimethylpyridine, imidazole, 1-methylimidazole, 1-methylpiperidine, 4-methylmorpholine, tetramethyl ethylenediamine, or one or more of para-dimethylaminopyridine. When the diester is specialized into a dihalide, the reaction reagent H is selected from magnesium chloride, magnesium bromide, magnesium iodide, zinc chloride, zinc bromide, zinc iodide, lithium chloride, lithium bromide, lithium iodide, sodium chloride, sodium bromide, sodium iodide, potassium chloride, potassium bromide, potassium iodide, tetrabutylammonium chloride, tetrabutylammonium bromide, tetrabutylammonium iodide, tetraethylammonium bromide, tetraethylammonium iodide, benzyltriethylammonium bromide, benzyltriethylammonium iodide, etc.

Step four is the method for synthesizing compound V. Compound III, compound IV, and reagent J are added to solvent I to react and produce compound V. The reaction temperature ranges from −30 degrees to 80 degrees, with the preferred temperature being 50 to 60 degrees. This method introduces a novel intermediate design, as shown in the diagram below, which changes the original synthesis approach and breaks the limitations of traditional reaction methods, effectively addressing the shortcomings of the previous method.

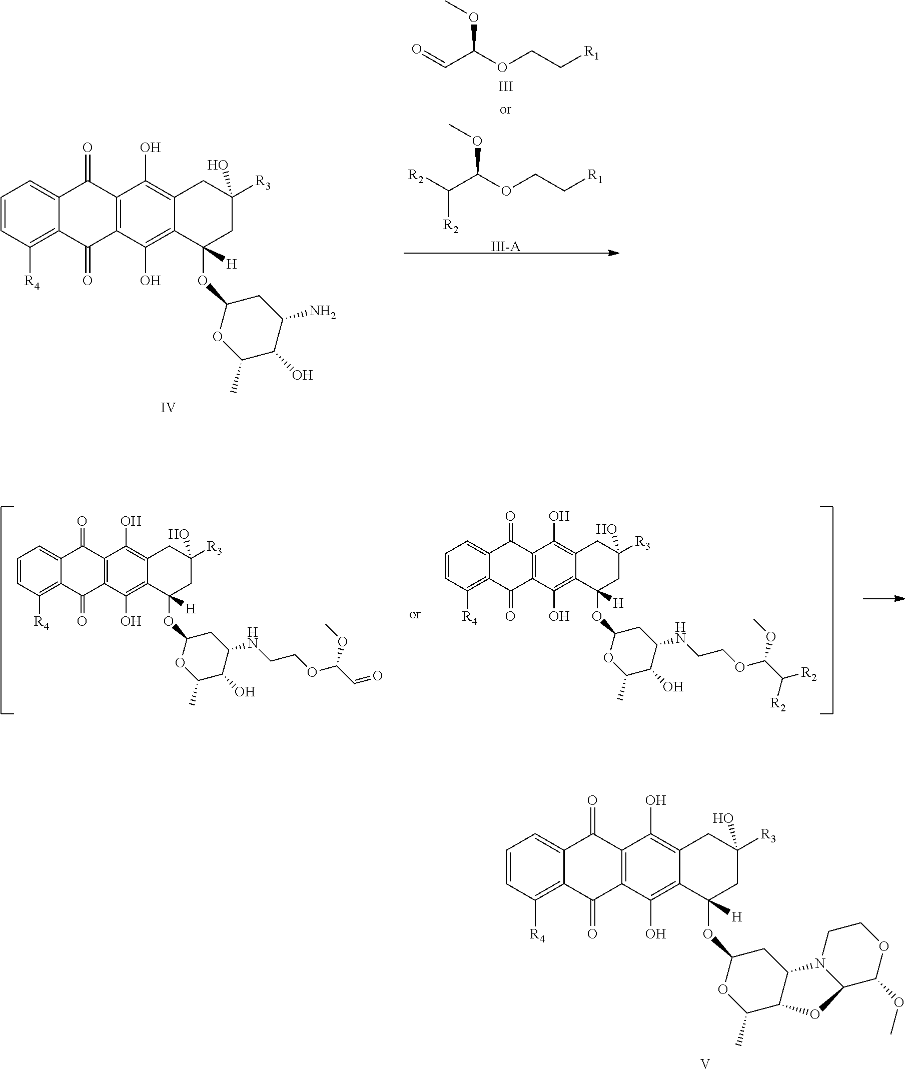

Solvent I is selected from one or more of dichloromethane, 1,2-dichloroethane, chloroform, carbon tetrachloride, methyl tert-butyl ether, tetrahydrofuran, 2-methyltetrahydrofuran, isopropyl ether, diethyl ether, ethylene glycol dimethyl ether, acetonitrile, acetone, ethyl acetate, methyl acetate, isopropyl acetate, 1,4-dioxane, benzene, toluene, N,N-dimethylformamide, N,N-dimethylacetamide; preferably dichloromethane, acetonitrile, and N,N-dimethylformamide.

Reaction reagents J are selected from sodium bicarbonate, sodium carbonate, potassium bicarbonate, potassium carbonate, cesium carbonate, potassium phosphate, sodium phosphate, dipotassium hydrogen phosphate, disodium hydrogen phosphate, sodium hydroxide, potassium hydroxide, triethylamine, N,N-diisopropylethylamine, pyridine, 2-methylpyridine, 2,6-dimethylpyridine, imidazole, 1-methylimidazole, 1-methylpiperidine, 4-methylmorpholine, tetramethyl ethylenediamine, piperidine, and other compounds.

The structure of compound IV in this patent is as follows:

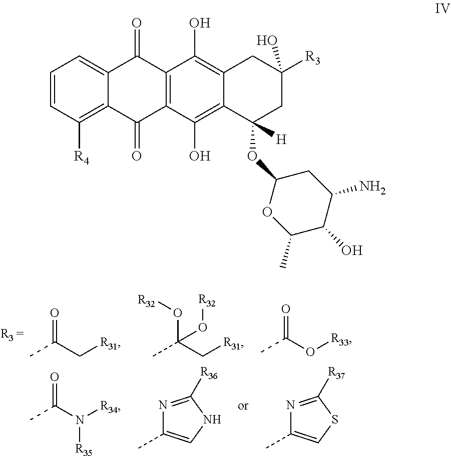

R4 can be hydrogen (H), fluorine (F), chlorine (Cl), bromine (Br), iodine (I), hydroxyl (OH), amino (NH2), hydrazino (NHNH2), thiol (SH), trifluoromethanesulfonyloxy, methanesulfonyloxy, benzenesulfonyloxy, para-toluenesulfonyloxy, para-fluorobenzenesulfonyloxy, ortho-nitrobenzenesulfonyloxy, para-nitrobenzenesulfonyloxy, acetyloxy, trifluoroacetyloxy, benzoyloxy, pivaloyloxy, benzyloxy, para-methoxybenzyloxy, 3,4-dimethoxybenzyloxy, triphenylmethoxy, 2-tetrahydropyranyloxy, methoxymethoxy, 2-ethoxyethoxy, 2-(trimethylsilyl)ethoxymethoxy, allyloxy, benzyloxycarbonyloxy, 4-nitrobenzyloxycarbonyloxy, pentafluorobenzyloxycarbonyloxy, trimethylsiloxy, tert-butyldimethylsiloxy, tert-butyldiphenylsiloxy, diethylisopropylsiloxy, triisopropylsiloxy, triphenylsiloxy, trifluoromethanesulfonylamino, methanesulfonylamino, benzenesulfonylamino, para-toluenesulfonylamino, para-fluorobenzenesulfonylamino, ortho-nitrobenzenesulfonylamino, para-nitrobenzenesulfonylamino, 2-(trimethylsilyl)ethanesulfonyl, acetylamino, trifluoroacetylamino, benzoylamino, pivaloylamino, ortho-phthaloylhydrazino, benzylamino, para-methoxybenzylamino, 3,4-dimethoxybenzylamino, allylamino, triphenylmethylamino, methoxycarbonylamino, ethoxycarbonylamino, benzyloxycarbonylamino, tert-butyloxycarbonylamino, fluorenylmethoxycarbonylamino, allyloxycarbonylamino, trimethylsilyl ethoxycarbonylamino, trichloroethoxycarbonylamino, various oxygen alkyl derivatives, and amine alkyl derivatives, etc.

Among them, R31 represents hydrogen (H), fluorine (F), chlorine (Cl), bromine (Br), iodine (I), hydroxyl (OH), amino (NH2), hydrazino (NHNH2), thiol (SH), trifluoromethanesulfonyloxy, methanesulfonyloxy, benzenesulfonyloxy, para-toluenesulfonyloxy, para-fluorobenzenesulfonyloxy, ortho-nitrobenzenesulfonyloxy, para-nitrobenzenesulfonyloxy, acetyloxy, trifluoroacetyloxy, benzoyloxy, pivaloyloxy, benzylloxy, para-methoxybenzyloxy, 3,4-dimethoxybenzyloxy, triphenylmethyloxy, 2-tetrahydropyranyloxy, methoxymethoxy, 2-ethoxyethoxy, 2-(trimethylsilyl)ethoxymethoxy, allyloxy, phenoxycarbonyloxy, 4-nitrophenoxycarbonyloxy, pentafluorophenoxycarbonyloxy, trimethoxysilyl, tert-butyldimethylsilyl, tert-butyldiphenylsilyl, diethylisopropylsilyl, triisopropylsilyl, triphenylsilyl, trifluoromethanesulfonylamino, methanesulfonylamino, benzenesulfonylamino, para-toluenesulfonylamino, para-fluorobenzenesulfonylamino, ortho-nitrobenzenesulfonylamino, para-nitrobenzenesulfonylamino, 2-(trimethylsilyl)ethylsulfonyl, acetylamino, trifluoroacetylamino, benzoylamino, pivaloylamino, ortho-phthaloylhydrazino, benzylamino, para-methoxybenzylamino, 3,4-dimethoxybenzylamino, allylamino, triphenylmethylamino, methoxycarbonylamino, ethoxycarbonylamino, benzylcarbonylamino, tert-butyloxycarbonylamino, fluorenylmethoxycarbonylamino, allyloxycarbonylamino, trimethylsilyl ethoxycarbonylamino, trichloroethoxycarbonylamino, various oxygen alkyl linking groups and amine alkyl linking groups, etc.

Among them, R32 is a silyl protecting group derived from methanol, ethanol, 1-propanol, 1-butanol, 1-pentanol, ethylene glycol, 1,2-propanediol, 1,3-propanediol, 2-methyl-1,3-propanediol, 2-ethyl-1,3-propanediol, 2-n-propyl-1,3-propanediol, 2-ethyl-2-methyl-1,3-propanediol, 2-isopropylpropane-1,3-diol, 2-tert-butylpropane-1,3-diol, 2-methoxy-1,3-propanediol, 2,2-dimethyl-1,3-propanediol, 2,2-ethyl-1,3-propanediol, 1,1-cyclohexanediol, 1,1-di(hydroxymethyl)cyclopentane, 1,1-di(hydroxymethyl)cyclohexane, 1,1-di(hydroxymethyl)cycloheptane, 1,3-butanediol, 2-methyl-1,3-butanediol, 1,3-pentanediol, 2,4-pentanediol, pinacol, hydrogenated hydroquinone, resorcinol, etc. R33, R34, R35, R36, and R37 represent hydrogen (H), methyl, ethyl, propyl, isopropyl, tert-butyl, benzyl, allyl, para-methoxybenzyl, 3,4-dimethoxybenzyl, triphenylmethyl, various oxygen alkyl derivatives, and amine alkyl derivatives, etc.

The typical structure of compound IV is shown below:

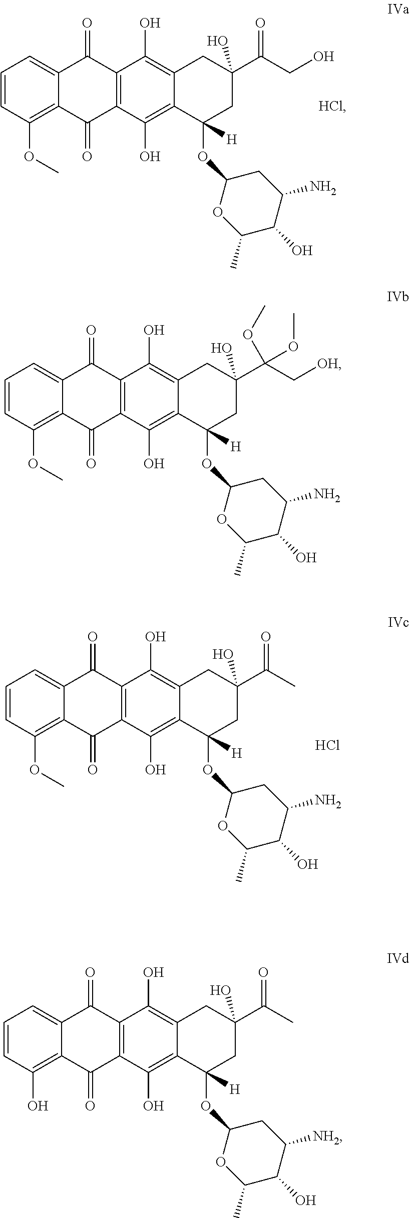 [SMILES: COc1cccc2c1C(=O)c1c(O)c3c(c(O)c1C2=O)C[C@@](O)(C(=O)CO)C[C@@H]3O[C@H]1C[C@H](N)[C@H](O)[C@H](C)O1 ; "HCl," ; COc1cccc2c1C(=O)c1c(O)c3c(c(O)c1C2=O)C[C@@](O)(C(CO)(OC)OC)C[C@@H]3O[C@H]1C[C@H](N)[C@H](O)[C@H](C)O1 ; COc1cccc2c1C(=O)c1c(O)c3c(c(O)c1C2=O)C[C@@](O)(C(C)=O)C[C@@H]3O[C@H]1C[C@H](N)[C@H](O)[C@H](C)O1 ; Cl ; CC(=O)[C@]1(O)Cc2c(O)c3c(c(O)c2[C@@H](O[C@H]2C[C@H](N)[C@H](O)[C@H](C)O2)C1)C(=O)c1c(O)cccc1C3=O]

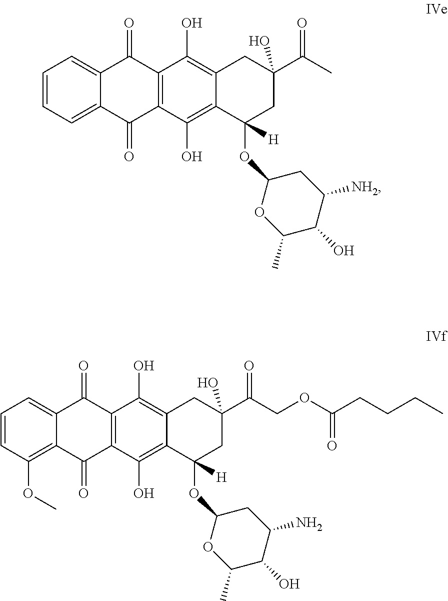 [SMILES: CC(=O)[C@]1(O)Cc2c(O)c3c(c(O)c2[C@@H](O[C@H]2C[C@H](N)[C@H](O)[C@H](C)O2)C1)C(=O)c1ccccc1C3=O ; CCCCC(=O)OCC(=O)[C@]1(O)Cc2c(O)c3c(c(O)c2[C@@H](O[C@H]2C[C@H](N)[C@H](O)[C@H](C)O2)C1)C(=O)c1c(OC)cccc1C3=O]

The structure of compound V in this patent is as follows:

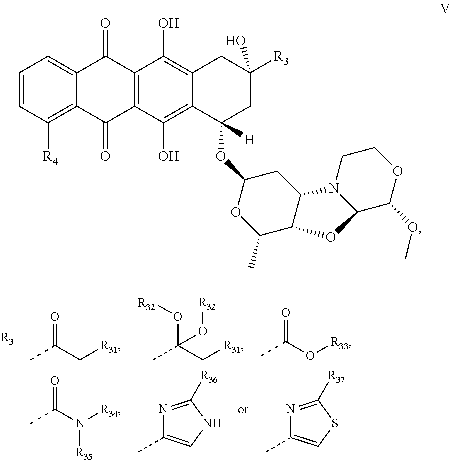

R4 includes hydrogen (H), fluorine (F), chlorine (Cl), bromine (Br), iodine (I), hydroxyl group (OH), amino group (NH2), hydrazine group (NHNH2), thiol group (SH), trifluoromethanesulfonyloxy group, methanesulfonyloxy group, benzenesulfonyloxy group, para-toluenesulfonyloxy group, para-fluorobenzenesulfonyloxy group, ortho-nitrobenzenesulfonyloxy group, para-nitrobenzenesulfonyloxy group, acetyloxy group, trifluoroacetyloxy group, benzoyloxy group, pivaloyloxy group, benzyl oxy group, para-methoxybenzyl oxy group, 3,4-dimethoxybenzyl oxy group, triphenylmethoxy group, 2-tetrahydropyranyl oxy group, methoxymethoxy group, 2-ethoxyethoxy group, 2-(trimethylsilyl)ethoxymethoxy group, allyloxy group, benzoyloxy carbonyloxy group, 4-nitrobenzoyloxy carbonyloxy group, pentafluorobenzoyloxy carbonyloxy group, trimethylsiloxy group, tert-butyldimethylsiloxy group, tert-butyldiphenylsiloxy group, diethylisopropylsiloxy group, triisopropylsiloxy group, triphenylsiloxy group, trifluoromethanesulfonyl amino group, methanesulfonyl amino group, benzenesulfonyl amino group, para-toluenesulfonyl amino group, para-fluorobenzenesulfonyl amino group, ortho-nitrobenzenesulfonyl amino group, para-nitrobenzenesulfonyl amino group, 2-(trimethylsilyl)ethylsulfonyl, acetylamino group, trifluoroacetylamino group, benzoylamino group, pivaloylamino group, ortho-phenylene dimethylene imino group, benzylamino group, para-methoxybenzylamino group, 3,4-dimethoxybenzylamino group, allylamino group, triphenylmethylamino group, methoxycarbonylamino group, ethoxycarbonylamino group, benzoxycarbonylamino group, tert-butoxycarbonylamino group, fluorenylmethoxycarbonylamino group, allyloxycarbonylamino group, trimethylsilyl ethoxycarbonylamino group, trichloroethoxycarbonylamino group, various derivatives of alkoxyl and amine alkyl connecting groups, etc.

Among them, R31 represents hydrogen H, fluorine F, chlorine Cl, bromine Br, iodine I, hydroxyl OH, amino NH2, hydrazine NHNH2, thiol SH, trifluoromethanesulfonyloxy, methanesulfonyloxy, benzenesulfonyloxy, para-toluenesulfonyloxy, para-fluorobenzenesulfonyloxy, ortho-nitrobenzenesulfonyloxy, para-nitrobenzenesulfonyloxy, acetyloxy, trifluoroacetyloxy, benzoyloxy, pivaloyloxy, benzyloxy, para-methoxybenzyloxy, 3,4-dimethoxybenzyloxy, triphenylmethoxy, 2-tetrahydropyranyloxy, methoxymethoxy, 2-ethoxyethoxy, 2-(trimethylsilyl)ethoxymethoxy, allyloxy, phenoxycarbonyloxy, 4-nitrophenoxycarbonyloxy, pentafluorophenoxycarbonyloxy, trimethoxysilane, tert-butyldimethylsiloxy, tert-butyldiphenylsiloxy, diethylisopropylsiloxy, triisopropylsiloxy, triphenylsiloxy, trifluoromethanesulfonylamino, methanesulfonylamino, benzenesulfonylamino, para-toluenesulfonylamino, para-fluorobenzenesulfonylamino, ortho-nitrobenzenesulfonylamino, para-nitrobenzenesulfonylamino, 2-(trimethylsilyl)ethanesulfonyl, acetylamino, trifluoroacetylamino, benzoylamino, pivaloylamino, ortho-phenylenediamine, benzylamino, para-methoxybenzylamino, 3,4-dimethoxybenzylamino, allylamino, triphenylmethylamino, methoxycarbonylamino, ethoxycarbonylamino, benzyloxycarbonylamino, tert-butyloxycarbonylamino, fluorenylmethoxycarbonylamino, allyloxycarbonylamino, trimethylsilylethoxycarbonylamino, trichloroethoxycarbonylamino, various derivatives of alkoxy and alkylamine compounds, etc.

Among them, R32 is a tert-butoxycarbonyl protecting group, derived from methanol, ethanol, 1-propanol, 1-butanol, 1-pentanol, ethylene glycol, 1,2-propanediol, 1,3-propanediol, 2-methyl-1,3-propanediol, 2-ethyl-1,3-propanediol, 2-n-propyl-1,3-propanediol, 2-ethyl-2-methyl-1,3-propanediol, 2-isopropylpropane-1,3-diol, 2-tert-butylpropane-1,3-diol, 2-methoxy-1,3-propanediol, 2,2-dimethyl-1,3-propanediol, 2,2-ethyl-1,3-propanediol, 1,1-cyclopropanedimethanol, 1,1-di(hydroxymethyl)cyclobutane, 1,1-di(hydroxymethyl)cyclopentane, 1,1-di(hydroxymethyl)cyclohexane, 1,3-butanediol, 2-methyl-1,3-butanediol, 1,3-pentanediol, 2,4-pentanediol, pentaerythritol, hydrogenated hydroxystyrene, resorcinol, etc. R33, R34, R35, R36, and R37 are hydrogen, methyl, ethyl, propyl, isopropyl, tert-butyl, benzyl, allyl, para-methoxybenzyl, 3,4-dimethoxybenzyl, triphenylmethyl, various oxygenated alkyl linkers, and amine alkyl linkers, etc.

The typical structure of compound V is shown below:

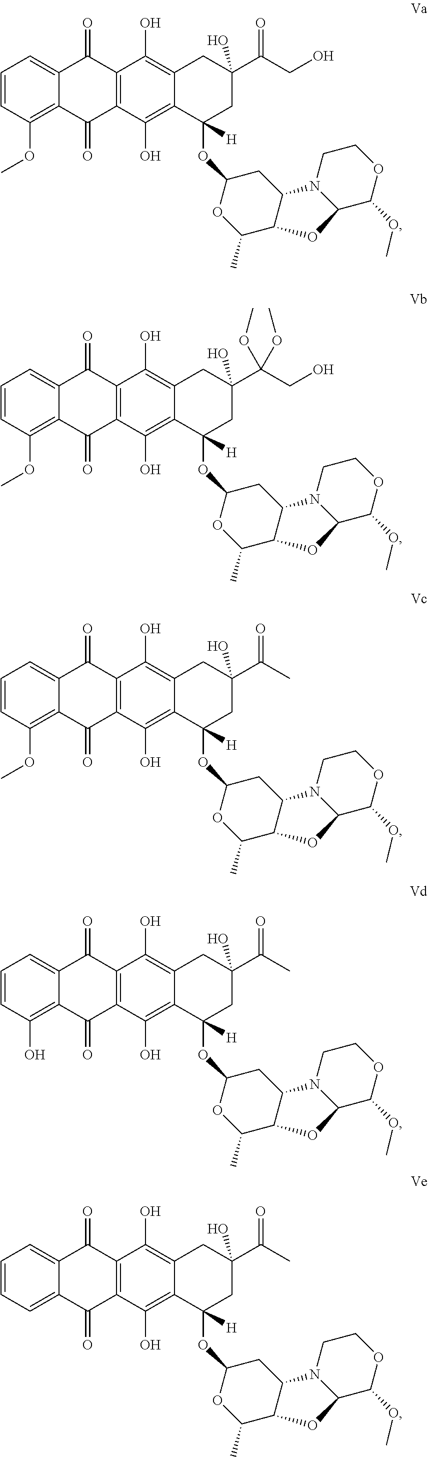

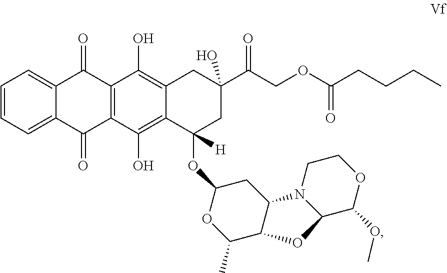 [SMILES: CCCCC(=O)OCC(=O)[C@]1(O)Cc2c(O)c3c(c(O)c2[C@@H](O[C@H]2C[C@H]4[C@H](O[C@@H]5[C@@H](OC)OCCN54)[C@H](C)O2)C1)C(=O)c1ccccc1C3=O]

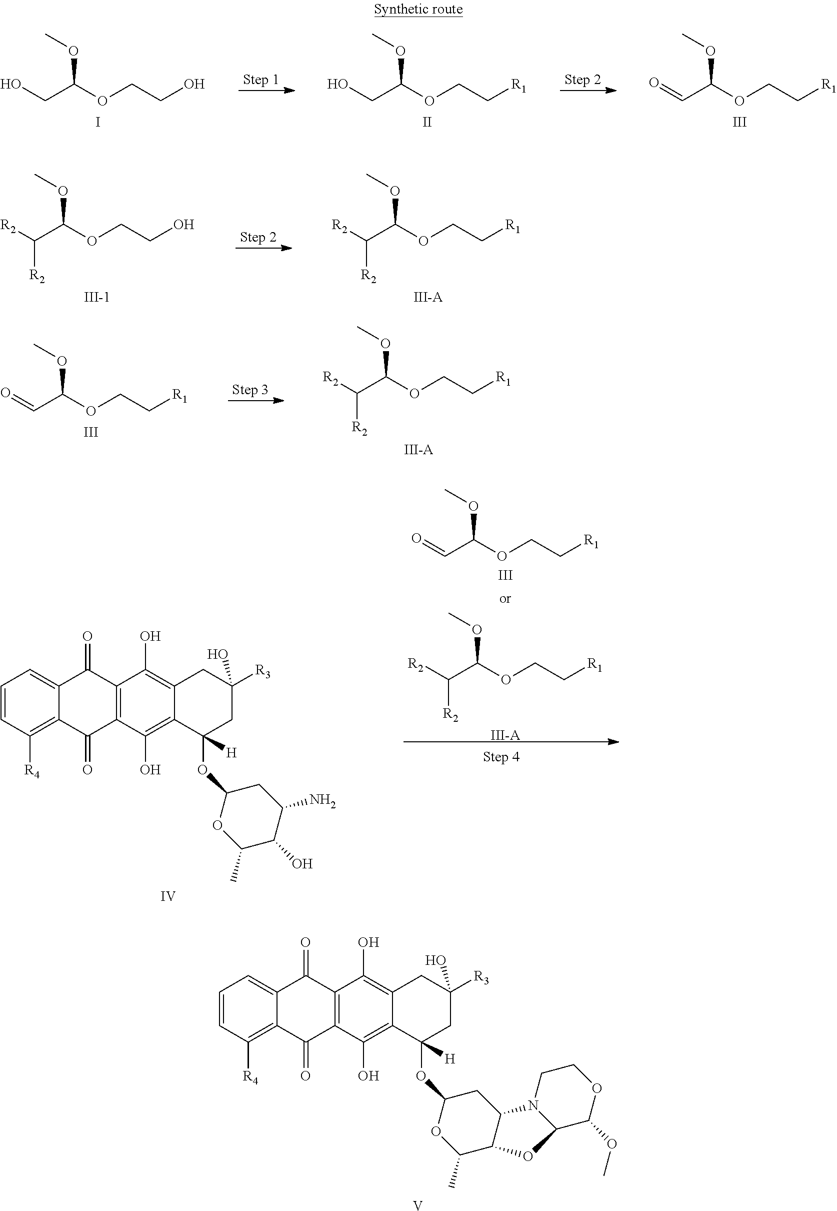

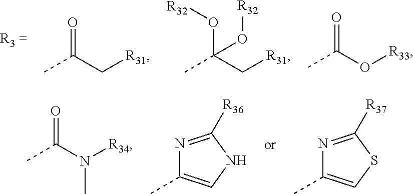

The raw materials and reagents involved in the reaction of the present invention can be purchased from the market or prepared according to the methods described in the literature. The monitoring and detection methods include TLC (thin-layer chromatography), LCMS (liquid chromatography-mass spectrometry), HPLC (high-performance liquid chromatography), and NMR (nuclear magnetic resonance) detection.

### Implementation Example One

Synthesis of Compound IIa: Methyl tert-butyl ether (140 mL), Compound I (13.62 g, 100 mmol), and 2,6-dimethylpyridine (21.41 g, 200 mmol) were added to a reaction flask and stirred. Trifluoromethanesulfonyl anhydride (31.10 g, 10 mmol) in methyl tert-butyl ether solution (60 mL) was added dropwise. After completion of the addition, the mixture was stirred for 30 minutes. Pure water (100 mL) was added to quench the reaction, and the organic phase was separated. The organic phase was sequentially washed with 5% sodium bicarbonate solution (100 mL) and saturated sodium chloride solution (100 mL), then dried over anhydrous sodium sulfate, filtered to remove the sodium sulfate, concentrated under reduced pressure, and purified by column chromatography using a gradient of n-hexane/ethyl acetate (10:1-4:1). The product was collected to yield a pale yellow liquid (19.72 g) with a 73% yield.

### Implementation Case Two

Synthesis of compound IIb: Dichloromethane (140 mL) was added to a reaction flask, followed by compound I (13.62 g, 100 mmol), N,N-dimethylpyridin-4-amine (1.22 g, 10 mmol), and triethylamine (12.12 g, 120 mmol). A solution of p-toluenesulfonyl chloride (20.04 g, 105 mmol) in dichloromethane (60 mL) was added dropwise with stirring. After the addition was complete, the mixture was stirred for 60 minutes. Pure water (100 mL) was added to quench the reaction, and the organic phase was separated. The organic phase was sequentially washed with 5% sodium bicarbonate solution (100 mL) and saturated sodium chloride solution (100 mL), dried over anhydrous sodium sulfate, filtered to remove the sodium sulfate, concentrated under reduced pressure, purified by column chromatography using a gradient elution of n-heptane/ethyl acetate (10:1-4:1), and the product was collected. A pale yellow liquid was obtained with a yield of 89%, weighing 25.72 g.

### Implementation Case Three

Synthesis of Compound IIc: Methyl tert-butyl ether (200 mL) and Compound I (13.62 g, 100 mmol) were added to a reaction flask and stirred. Phosphorus tribromide (5.43 g, 20 mmol) was added dropwise. After the addition was complete, the mixture was stirred at 0° C. for 30 minutes. 2,6-dimethylpyridine (2.11 g, 20 mmol) was then added dropwise. The mixture was concentrated under reduced pressure, purified by column chromatography using n-hexane/ethyl acetate (10:1-4:1) as the eluent. The product was collected as a colorless liquid, yielding 6.91 g with a 68% yield (based on phosphorus tribromide).

### Implementation Example Four

Synthesis of compound IIId: Compound I (13.62 g, 100 mmol), 2-methyl tetrahydrofuran (150 mL, water content less than 200 ppm), triphenylphosphine (28.92 g, 110 mmol), and imidazole (15.04 g, 220 mmol) were added to a reaction flask and stirred. Iodine (27.93 g, 110 mmol) was added dropwise under stirring. The mixture was stirred for 60 minutes. The reaction was quenched with a 5% sodium thiosulfate solution, turning the system colorless. Ethyl acetate was added for extraction, and the organic phase was separated. The organic phase was washed successively with a 5% sodium bicarbonate solution (100 mL) and saturated sodium chloride solution (100 mL), dried over anhydrous sodium sulfate, filtered to remove the sodium sulfate, concentrated under reduced pressure, and purified by column chromatography using a gradient of n-heptane/ethyl acetate (10:1 to 4:1). The product was collected, concentrated to dryness, yielding a colorless liquid of 16.93 g with a 69% yield.

### Implementation Example Five

Synthesis of compound IIIa: Compound IIa (5.36 g, 20 mmol) and acetic anhydride (15 mL) were added to a reaction flask, followed by the addition of sodium nitrite (6.90 g, 100 mmol). The mixture was stirred for 3 hours. The reaction mixture was then poured into water (100 mL), and extracted with methyl tert-butyl ether (100 mL×3). The combined organic phases were dried over anhydrous sodium sulfate, filtered to remove the sodium sulfate, and concentrated under reduced pressure. The resulting residue was purified by column chromatography using a mixture of n-hexane/ethyl acetate (10:1 to 4:1) as eluent. The product was collected as a colorless liquid, yielding 4.76 g with a yield of 89%.

### Implementation Case Six

Synthesis of compound IIIb: Compound IIb (5.81 g, 20 mmol), dimethyl sulfoxide (9.38 g, 120 mmol), dichloroacetic acid (1.29 g, 10 mmol), and ethyl acetate (50 mL) were added to a reaction flask, followed by the gradual addition of dicyclohexylcarbodiimide (12.38 g, 60 mmol) with stirring for 3 hours. The mixture was filtered to remove solids, and the filtrate was concentrated under reduced pressure, purified by column chromatography using n-hexane/ethyl acetate=10:1-4:1 as eluent. The product was collected as a colorless liquid in a yield of 4.89 g, corresponding to 84%.

### Implementation Example Seven

Synthesis of compound IIIc: Dimethyl sulfoxide (4.69 g, 60 mmol) and dichloromethane (30 mL) were added to a reaction flask under argon protection and cooled to −70 to −60 degrees. A solution of trifluoromethanesulfonyl anhydride (7.56 g, 36 mmol) in DCM (30 mL) was added dropwise and stirred for 10 minutes. A solution of compound IIc (5.97 g, 30 mmol) in DCM (30 mL) was then added dropwise and stirred for 10 minutes. A solution of N,N-diisopropylethylamine (11.22 g, 87 mmol) in DCM (30 mL) was added dropwise and stirred for 10 minutes, followed by natural warming to room temperature. The reaction mixture was poured into saturated sodium bicarbonate solution (200 mL), and the organic phase was separated, dried over anhydrous sodium sulfate, filtered to remove the sodium sulfate, and the filtrate was concentrated under reduced pressure. The crude product was purified by column chromatography using a gradient elution of n-heptane/ethyl acetate (10:1 to 4:1). The collected product was concentrated below 30 degrees to yield a pale yellow liquid (5.49 g) with a yield of 92%.

### Implementation Case Eight

Synthesis of compound IIId: Compound IId (4.92 g, 20 mmol) was used in accordance with Example Seven to obtain a pale yellow liquid IIId with a yield of 88%.

### Implementation Example Nine

Synthesis of compounds III-Aa and III-Ab: Compound IIIa (13.31 g, 50 mmol), 2,6-dimethylpyridine (15.83 g, 60 mmol), and dichloromethane (150 mL) were added to a reaction flask, cooled to 0 degrees under argon protection. Trifluoromethanesulfonyl anhydride (31.1 g, 110 mmol) was added dropwise, maintaining the temperature at 0-10 degrees, and stirred for 5 hours. The reaction mixture was then sealed and placed in a refrigerator at 0 degrees for 48 hours, concentrated to dryness below 30 degrees, and the residue was extracted with pentane (3×100 mL). The pentane extract was washed with 1M hydrochloric acid solution (50 mL), saturated sodium bicarbonate solution (50 mL), and brine (50 mL). The organic layer was dried over anhydrous sodium sulfate, filtered to remove solids, concentrated below 30 degrees to dryness, yielding a pale yellow liquid of 22.87 g, with a yield of 83%. Product III-Aa is unstable, prone to darkening in color to yellow-brown or dark brown, and should be stored under argon protection at −20 degrees.

Add compound III-Aa (5.38 g, 10 mmol) and carbon disulfide (50 mL) to a reaction flask under argon protection and cool to 0 degrees. Add magnesium iodide (8.34 g, 30 mmol) at 0 degrees and stir for 3 hours, then concentrate to dryness below 30 degrees. Extract the residue with pentane (3×30 mL). Wash the pentane extract with saturated sodium bicarbonate solution (50 mL) and brine (50 mL). Dry over anhydrous sodium sulfate, filter through 200-300 mesh silica gel, rinse the filter cake with pentane (30 mL), and concentrate the filtrate below 30 degrees to dryness to obtain a pale yellow liquid of 4.11 g, with a yield of 85%. Product III-Ab is unstable and prone to darkening to yellow-brown or dark brown. Store under argon protection at −20 degrees.

### Implementation Example Ten

Synthesis of compound Va: Compound IIIa (5.33 g, 20 mmol), compound IVa (5.80 g, 10 mmol), and 1,2-dichloroethane (80 mL) were added to a reaction flask under argon protection and stirred. Diisopropylethylamine (3.23 g, 25 mmol) was added, and the mixture was stirred overnight at 60-65° C. The reaction was quenched with pure water (300 mL) and extracted with dichloromethane (200×2). The organic phase was separated, washed with 5% sodium bicarbonate solution (100 mL) and saturated sodium chloride solution (100 mL), dried over anhydrous sodium sulfate, filtered to remove the sodium sulfate, concentrated under reduced pressure, purified by column chromatography, and the product was collected. The red solid obtained was concentrated to dryness below 30° C., yielding 1.43 g (21% yield).

### Implementation Example Eleven

Synthesis of compound Va: The red solid compound Va was obtained with a yield of 18% by using compound IIIc and compound IVa as references, following the implementation example ten.

### Implementation Example Twelve

Synthesis of compound Vb: A red solid Va was obtained with a yield of 15% by using compound IIId and compound IVa, following the implementation of Example Ten.

### Implementation Example Thirteen

Synthesis of compound Vc: A red solid Va was obtained with a yield of 22% by using compound IIIb and compound IVc, following the implementation example ten.

### Implementation Example Four

Synthesis of compound Vd: A red solid Va was obtained with a yield of 20% by using compound IIIc (197 mg, 1.0 mmol) and compound IVd (257 mg, 0.5 mmol), following the procedure in Example Ten.

### Implementation Example Fifteen

Synthesis of compound Ve: A red solid Ve was obtained with a yield of 15% by using compound IIIa and compound IVe, following the implementation example ten.

### Implementation Example Sixteen

Synthesis of compound Vf: The red solid Vf was obtained with a yield of 18% by using compound IIId and compound IVf, following the procedures outlined in Example Ten.

### Implementation Example Seventeen

Synthesis of compound Vc: Red solid Vc was obtained with a yield of 13% by using compound III-Aa and compound IVc, following the implementation example ten.

### Implementation Example Eighteen

Synthesis of compound Vc: Red solid Vc was obtained with a yield of 15% by using compound III-Ab and compound IVc, following the example ten.

What is claimed is:

1. An intermediate compound for preparing a class of anthracene-derived toxins having the structural formula as following: 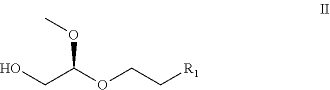
wherein R₁ is selected from the group consisting of trifluoromethanesulfonyloxy, methanesulfonyloxy, phenylsulfonyloxy, para-tolylsulfonyloxy, para-fluorophenylsulfonyloxy, ortho-nitrophenylsulfonyloxy, para-nitrophenylsulfonyloxy, chlorine (Cl), bromine (Br), and iodine (I).

2. The intermediate compound according to claim 1, wherein the structural formula is 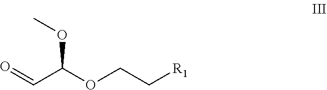
wherein R₁ is selected from the group consisting of rifluoromethanesulfonyloxy, methanesulfonyloxy, phenylsulfonyloxy, para-tolylsulfonyloxy, para-fluorophenylsulfonyloxy, ortho-nitrophenylsulfonyloxy, para-nitrophenylsulfonyloxy, chlorine (Cl), bromine (Br), and iodine (I).

3. The intermediate compound according to claim 2, wherein the structural formula is: 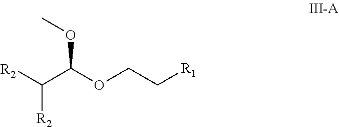
wherein
R₁ is selected from the group consisting of trifluoromethanesulfonyloxy, methanesulfonyloxy, phenylsulfonyloxy, para-tolylsulfonyloxy, para-fluorophenylsulfonyloxy, ortho-nitrophenylsulfonyloxy, para-nitrophenylsulfonyloxy, chlorine (Cl), bromine (Br), and iodine (I);
R₂ is an aldehyde or a diester or a dihalide; wherein the aldehyde is selected from the group consisting of methoxy, ethoxy, 1-propoxy, 1-butoxy, 1-pentoxy, ethylene dioxy, 2,3-butanedioxy, 1,2-diphenylethane dioxy, 1,2-propane dioxy, 1,3-propane dioxy, 2-methyl-1,3-propane dioxy, 2-ethyl-1,3-propane dioxy, 2-n-propyl-1,3-propane dioxy, 2-ethyl-2-methyl-1,3-propane dioxy, 2-isopropylpropane-1,3-dioxy, 2-tert-butylpropane-1,3-dioxy, 2-methoxy-1,3-propane dioxy, 2,2-dimethyl-1,3-propane dioxy, 2,2-ethyl-1,3-propane dioxy, 1,1-cyclopropane dimethoxy, 1,1-cyclobutane dimethoxy, 1,1-cyclopentane dimethoxy, 1,1-cyclohexane dimethoxy, 1,3-butanedioxy, 2-methyl-1,3-butanedioxy, 1,3-pentanedioxy and 2,4-pentanedioxy; the diester is selected from the group consisting of acetoxy, propionyloxy, butyryloxy, valeroyloxy, pivaloyloxy, trifluoroacetoxy, trifluoromethanesulfonyloxy, methanesulfonyloxy, phenylsulfonyloxy, para-tolylsulfonyloxy, para-fluorophenylsulfonyloxy, ortho-nitrophenylsulfonyloxy and para-nitrophenylsulfonyloxy; the dihalide is selected from the group consisting of chlorine (Cl), bromine (Br), and iodine (I).

4. A method for synthesizing the intermediate compound according to claim 3 comprising the following steps: 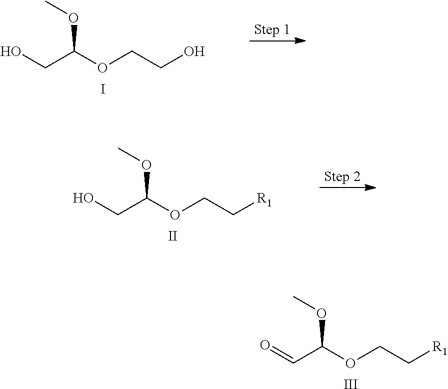
step 1: synthesizing the intermediate compound II from a compound I comprising the following protocols:
protocol 1: adding the compound I into a solvent A1, reacting with reaction reagent B1 and reaction reagent C1 to form a sulfonic acid ester (II) at −30 to 40° C.; wherein
the solvent A1 is one or more selected from the group consisting of dichloromethane, 1,2-dichloroethane, chloroform, carbon tetrachloride, methyl tert-butyl ether, tetrahydrofuran, 2-methyltetrahydrofuran, isopropyl ether, diethyl ether, ethylene glycol dimethyl ether, acetonitrile, acetone, ethyl acetate, methyl acetate, isopropyl acetate, 1,4-dioxane, benzene, toluene, N,N-dimethylformamide and N,N-dimethylacetamide;
the reaction reagent B1 is selected from the group consisting of trifluoromethanesulfonyl anhydride, trifluoromethanesulfonyl chloride, 2-[N,N-bis(trifluoromethanesulfonyl)amino]pyridine, N,N-bis(trifluoromethanesulfonyl)-5-chloro-2-aminopyridine, N,N-bis(trifluoromethanesulfonyl)aniline, methanesulfonyl anhydride, methanesulfonyl chloride, 4-methylbenzenesulfonyl anhydride, 4-methylbenzenesulfonyl chloride, benzenesulfonyl sulfide, benzenesulfonyl chloride, 4-fluorobenzenesulfonyl anhydride, 4-fluorobenzenesulfonyl chloride, ortho-nitrobenzenesulfonyl chloride, para-nitrobenzenesulfonyl chloride, ortho-nitrobenzenesulfonyl anhydride and para-nitrobenzenesulfonyl anhydride; and
the reaction reagent C1 is selected from potassium tert-butoxide, sodium tert-butoxide, butyllithium, diisopropylamine lithium, hexamethyldisilazane lithium, hexamethyldisilazane sodium, hexamethyldisilazane potassium, sodium hydride, triethylamine, N,N-diisopropylethylamine, pyridine, 2-methylpyridine, 2,6-dimethylpyridine, imidazole, 1-methylimidazole, 1-methylpiperidine, 4-methylmorpholine, tetramethyl ethylenediamine, or N,N-diisopropylamino pyridine;
protocol 2: adding compound I into solvent A2, reacting with reagents B2, C2, and D to form a mono-substituted halide (II) at −30 to 40° C.; wherein
the solvent A2 is one or more selected from the group consisting of dichloromethane, 1,2-dichloroethane, chloroform, carbon tetrachloride, methyl tert-butyl ether, tetrahydrofuran, 2-methyltetrahydrofuran, isopropyl ether, ether, ethylene glycol dimethyl ether, acetonitrile, acetone, ethyl acetate, methyl acetate, isopropyl acetate, 1,4-dioxane, benzene, toluene, N,N-dimethylformamide and N,N-dimethylacetamide;
the reagent B2 is selected from the group consisting of carbon tetrachloride, hexachloroacetone, N-chlorosuccinimide, liquid bromine, carbon tetrabromide, chlorobromomethane, N-bromosuccinimide, iodomethane, iodoethane, diiodomethane, iodoform, N-iodosuccinimide, and iodine;
the reagent C2 is selected from the group consisting of triphenylphosphine or triphenylphosphite; and
the reagent D is selected from triethylamine, N,N-diisopropylethylamine, pyridine, 2-methylpyridine, 2,6-dimethylpyridine, imidazole, 1-methylimidazole, 1-methylpiperidine, 4-methylmorpholine, tetramethyl ethylenediamine, and piperidine;
protocol 3: adding compound I to solvent A3, reacting with reagents B3 and C3 to form a mono-substituted halide (II) at −30 to 40° C.; wherein
the solvent A3 is one or more selected from the group consisting of dichloromethane, 1,2-dichloroethane, chloroform, carbon tetrachloride, methyl tert-butyl ether, tetrahydrofuran, 2-methyltetrahydrofuran, isopropyl ether, diethyl ether, ethylene glycol dimethyl ether, acetonitrile, acetone, ethyl acetate, methyl acetate, isopropyl acetate, 1,4-dioxane, benzene, toluene, N,N-dimethylformamide and N,N-dimethylacetamide;
the reagent B3 is selected from the group consisting of sulfonyl chloride, phosphorus trichloride, phosphorus oxychloride, phosphorus pentachloride, phosphorus tribromide, phosphorus oxybromide, and phosphorus pentabromide; and
the reagent C3 is selected from the group consisting triethylamine, N,N-diisopropylethylamine, pyridine, 2-methylpyridine, 2,6-dimethylpyridine, imidazole, 1-methylimidazole, 1-methylpiperidine, 4-methylmorpholine, tetramethyl ethylenediamine, and piperidine;
step 2: synthesizing the intermediate compound III from the intermediate compound II comprising the following steps:
adding the intermediate compound II in solvent E, reacting with a reagent F to form the intermediate compound III at −70° C. to 40° C.; wherein
the solvent E is one or more selected from the group consisting of dichloromethane, 1,2-dichloroethane, chloroform, carbon tetrachloride, methyl tert-butyl ether, tetrahydrofuran, 2-methyltetrahydrofuran, isopropyl ether, ether, ethylene glycol dimethyl ether, acetonitrile, acetone, ethyl acetate, methyl acetate, isopropyl acetate, 1,4-dioxane, benzene, toluene, N,N-dimethylformamide, and N,N-dimethylacetamide; and
the reagent F is selected from the group consisting of 2-iodobenzoyl benzoic acid, Jones reagent, Collins reagent, pyridinium chlorochromate, pyridinium chlorochromate, Dess-Martin oxidant, m-chloroperbenzoic acid, acyl chloride/dimethyl sulfoxide/triethylamine combination, trifluoromethanesulfonyl anhydride/dimethyl sulfoxide/N,N-diisopropylethylamine combination, sodium nitrite/acetic anhydride combination, and hexamethylphosphoramide/dimethyl sulfoxide/dichloroacetic acid combination.

5. The method according to claim 4, wherein the method further comprises the following steps: 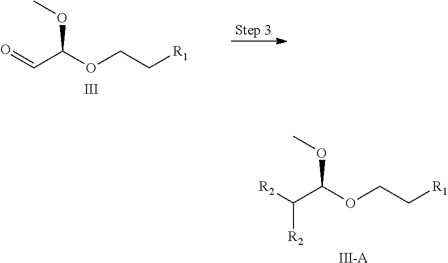
compound III reacting with reagent H in solvent G to generate the intermediate compound III-A at −70° C. to 70° C.; wherein the reaction reagent H is a single reagent or a combination of multiple reagents, and the reaction temperature ranges from −70° C. to 70° C.;
the solvent G is one or more selected from the group consisting of dichloromethane, 1,2-dichloroethane, chloroform, carbon tetrachloride, methyl tert-butyl ether, tetrahydrofuran, 2-methyltetrahydrofuran, isopropyl ether, diethyl ether, ethylene glycol dimethyl ether, methanol, ethanol, 1-propanol, acetonitrile, acetone, butanone, ethyl acetate, methyl acetate, isopropyl acetate, 1,4-dioxane, benzene, toluene, N,N-dimethylformamide, N,N-dimethylacetamide, and carbon disulfide;
the reagent H is one or more selected from the group consisting of trimethyl orthoformate, triethyl orthoformate, 1-butanol, 1-pentanol, ethylene glycol, 1,2-propanediol, 1,3-propanediol, 2-methyl-1,3-propanediol, 2-ethyl-1,3-propanediol, 2-n-propyl-1,3-propanediol, 2-ethyl-2-methyl-1,3-propanediol, 2-isopropylpropane-1,3-diol, 2-tert-butylpropane-1,3-diol, 2-methoxy-1,3-propanediol, 2,2-dimethyl-1,3-propanediol, 2,2-ethyl-1,3-propanediol, 1,1-cyclohexanediol, 1,1-dihydroxymethylcyclobutane, 1,1-dihydroxymethylcyclopentane, 1,1-dihydroxymethylcyclohexane, 1,3-butanediol, 2-methyl-1,3-butanediol, 1,3-pentanediol, 2,4-pentanediol, and pentaerythritol when the aldehyde group of the compound III is converted to an acetal;
the reaction reagent H is one or more selected from the group consisting of acetic anhydride, propionic anhydride, butyric anhydride, valeric anhydride, pivalic anhydride, trifluoroacetic anhydride, trifluoromethanesulfonic anhydride, methanesulfonic anhydride, benzenesulfonic anhydride, p-toluenesulfonic anhydride, p-fluorobenzenesulfonic anhydride, o-nitrobenzenesulfonic anhydride, p-nitrobenzenesulfonic anhydride, triethylamine, N,N-diisopropylethylamine, pyridine, 2-methylpyridine, 2,6-dimethylpyridine, imidazole, 1-methylimidazole, 1-methylpiperidine, 4-methylmorpholine, tetramethyl ethylenediamine, or piperidine. When the diester is converted to a dihalide, the reaction reagent H is selected from magnesium chloride, magnesium bromide, magnesium iodide, zinc chloride, zinc bromide, zinc iodide, lithium chloride, lithium bromide, lithium iodide, sodium chloride, sodium bromide, sodium iodide, potassium chloride, potassium bromide, potassium iodide, tetra-n-butylammonium chloride, tetra-n-butylammonium bromide, tetra-n-butylammonium iodide, tetraethylammonium bromide, tetraethylammonium iodide, benzyltriethylammonium bromide, and benzyltriethylammonium iodide when the aldehyde group of the compound III is converted to a diester.

6. The method according to claim 5, wherein the method further comprises the following steps: 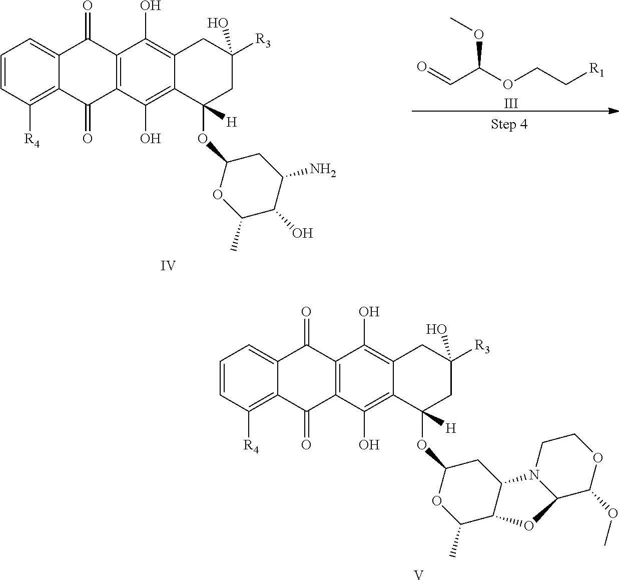
adding the intermediate compound III, compound IV and reagent J in solvent I for reaction to generate compound V at −30° C. to 80° C.; wherein 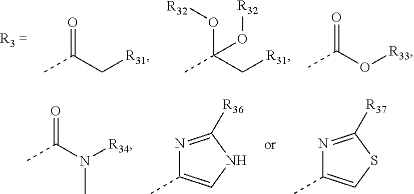
the Solvent I is one or more selected from the group consisting of dichloromethane, 1,2-dichloroethane, chloroform, carbon tetrachloride, methyl tert-butyl ether, tetrahydrofuran, 2-methyltetrahydrofuran, isopropyl ether, ethyl ether, ethylene glycol dimethyl ether, acetonitrile, N,N-dimethylformamide, N,N-dimethylacetamide; reagent J is selected from triethylamine, N,N-diisopropylethylamine, pyridine, 2-methylpyridine, 2,6-dimethylpyridine, imidazole, 1-methylimidazole, 1-methylpiperidine, 4-methylmorpholine, tetramethyl ethylenediamine, and piperidine;
wherein, R₄ is selected from the group consisting of hydrogen H, fluorine F, chlorine Cl, bromine Br, iodine I, hydroxyl OH, amino NH2, hydrazino NHNH2, mercapto SH, trifluoromethanesulfonyloxy, methanesulfonyloxy, benzenesulfonyloxy, para-toluenesulfonyloxy, para-fluorobenzenesulfonyloxy, ortho-nitrobenzenesulfonyloxy, para-nitrobenzenesulfonyloxy, acetyloxy, trifluoroacetyloxy, benzoyloxy, tigloyloxy, benzyl oxy, para-methoxybenzyl oxy, 3,4-dimethoxybenzyl oxy, triphenylmethoxy, 2-tetrahydropyranyl oxy, methoxymethoxy, 2-ethoxyethoxy, 2-(trimethylsilyl)ethoxy methoxy, allyloxy, benzoyloxy carbonyloxy, 4-nitrobenzyloxy carbonyloxy, pentafluorobenzyloxy carbonyloxy, trimethylsiloxy, tert-butyldimethylsiloxy, tert-butyldiphenylsiloxy, diethylisopropylsiloxy, triisopropylsiloxy, triphenylsiloxy, trifluoromethanesulfonylamino, methanesulfonylamino, benzenesulfonylamino, para-toluenesulfonylamino, para-fluorobenzenesulfonylamino, ortho-nitrobenzenesulfonylamino, para-nitrobenzenesulfonylamino, 2-(trimethylsilyl)ethylsulfonyl, acetylamino, trifluoroacetylamino, benzoylamino, tigloylamino, ortho-phenylenediacetylamino, benzylamino, para-methoxybenzylamino, 3,4-dimethoxybenzylamino, allylamino, triphenylmethylamino, methoxycarbonylamino, ethoxycarbonylamino, benzylcarbonylamino) cyclohexane, 1,3-butanediol, 2-methyl-1,3-butanediol, 1,3-pentanediol, and 2,4-pentanediol;
R₃₁ is selected from the group consisting of hydrogen H, fluorine F, chlorine Cl, bromine Br, iodine I, hydroxyl OH, amino NH2, hydrazino NHNH2, thiol SH, trifluoromethanesulfonyloxy, methanesulfonyloxy, benzenesulfonyloxy, para-toluenesulfonyloxy, para-fluorobenzenesulfonyloxy, ortho-nitrobenzenesulfonyloxy, para-nitrobenzenesulfonyloxy, acetyloxy, trifluoroacetyloxy, benzoyloxy, pivaloyloxy, benzyloxy, para-methoxybenzyloxy, 3,4-dimethoxybenzyloxy, triphenylmethoxy, 2-tetrahydropyranyloxy, methoxymethoxy, 2-ethoxyethoxy, 2-(trimethylsilyl)ethoxymethoxy, allyloxy, benzyloxycarbonyloxy, 4-nitrobenzyloxycarbonyloxy, pentafluorobenzyloxycarbonyloxy, trimethylsiloxy, tert-butyldimethylsiloxy, tert-butyldiphenylsiloxy, diethylisopropylsiloxy, triisopropylsiloxy, triphenylsiloxy, trifluoromethanesulfonylamino, methanesulfonylamino, benzenesulfonylamino, para-toluenesulfonylamino, para-fluorobenzenesulfonylamino, ortho-nitrobenzenesulfonylamino, para-nitrobenzenesulfonylamino, 2-(trimethylsilyl)ethylsulfonyl, acetylamino, trifluoroacetylamino, benzoylamino, pivaloylamino, ortho-tolylureido, benzylamino, para-methoxybenzylamino, 3,4-dimethoxybenzylamino, allylamino, triphenylamino, methoxycarbonylamino, ethoxycarbonylamino, benzyloxycarbonylamino, tert-butyloxycarbonylamino, fluorenyloxycarbonylamino, allyloxycarbonylamino, trimethylsiloxyethoxycarbonylamino, and trichloroethoxycarbonylamino;
R₃₂ is a ketone protecting group derived from methanol, ethanol, 1-propanol, 1-butanol, 1-pentanol, ethylene glycol, 1,2-propanediol, 1,3-propanediol, 2-methyl-1,3-propanediol, 2-ethyl-1,3-propanediol, 2-n-propyl-1,3-propanediol, 2-ethyl-2-methyl-1,3-propanediol, 2-isopropylpropane-1,3-diol, 2-tert-butylpropane-1,3-diol, 2-methoxy-1,3-propanediol, 2,2-dimethyl-1,3-propanediol, 2,2-ethyl-1,3-propanediol, 1,1-cyclohexanedimethanol, 1,1-bis(hydroxymethyl)cyclobutane, 1,1-bis(hydroxymethyl)cyclopentane, 1,1-bis(hydroxymethyl)cyclohexane, 1,3-butanediol, 2-methyl-1,3-butanediol, 1,3-pentanediol, or 2,4-pentanediol; and
R₃₃, R₃₄, R₃₅, R₃₆, and R₃₇ are hydrogen H, methyl, ethyl, propyl, isopropyl, tert-butyl, benzyl, allyl, para-methoxybenzyl, 3,4-dimethoxybenzyl, or triphenylmethyl, respectively.

7. The method according to claim 6, wherein the method further comprises the following steps: 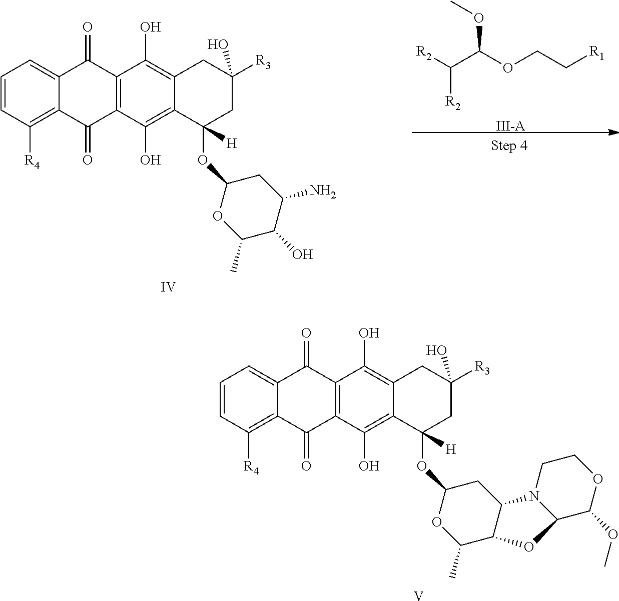
adding the intermediate compound III-A, compound IV, and reagent J in solvent I for reaction to generate compound V at −30° C. to 80° C.; wherein 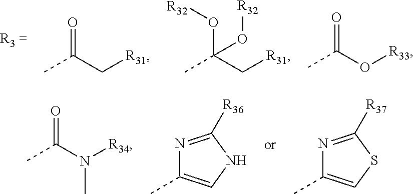
the solvent I is one or more selected from the group consisting of dichloromethane, 1,2-dichloroethane, chloroform, carbon tetrachloride, methyl tert-butyl ether, tetrahydrofuran, 2-methyltetrahydrofuran, isopropyl ether, ether, ethylene glycol dimethyl ether, acetonitrile, N,N-dimethylformamide, N,N-dimethylacetamide, or a combination thereof; reagent J is selected from triethylamine, N,N-diisopropylethylamine, pyridine, 2-methylpyridine, 2,6-dimethylpyridine, imidazole, 1-methylimidazole, 1-methylpiperidine, 4-methylmorpholine, tetramethyl ethylenediamine, or para-dimethylaminopyridine;
wherein R4 is hydrogen H, fluorine F, chlorine Cl, bromine Br, iodine I, hydroxyl OH, amino NH2, hydrazino NHNH2, thiol SH, trifluoromethanesulfonyloxy, methanesulfonyloxy, benzenesulfonyloxy, para-toluenesulfonyloxy, para-fluorobenzenesulfonyloxy, ortho-nitrobenzenesulfonyloxy, para-nitrobenzenesulfonyloxy, acetyloxy, trifluoroacetyloxy, benzoyloxy, tert-butoxycarbonyloxy, benzyl oxy, para-methoxybenzyl oxy, 3,4-dimethoxybenzyl oxy, triphenylmethoxy, 2-tetrahydropyranyl oxy, methoxymethoxy, 2-ethoxyethoxy, 2-(trimethylsilyl)ethoxy methoxy, allyloxy, benzyloxycarbonyloxy, 4-nitrobenzyloxycarbonyloxy, pentafluorobenzyloxycarbonyloxy, trimethylsiloxy, tert-butyldimethylsiloxy, tert-butyldiphenylsiloxy, diethylisopropylsiloxy, triisopropylsiloxy, triphenylsiloxy, trifluoromethanesulfonylamino, methanesulfonylamino, benzenesulfonylamino, para-toluenesulfonylamino, para-fluorobenzenesulfonylamino, ortho-nitrobenzenesulfonylamino, para-nitrobenzenesulfonylamino, 2-(trimethylsilyl)ethylsulfonyl, acetylamino, trifluoroacetylamino, benzoylamino, tert-butoxycarbonylamino, ortho-benzyldimide, benzylamino, para-methoxybenzylamino, 3,4-dimethoxybenzylamino, allylamino, triphenylmethylamino, methoxycarbonylamino, ethoxycarbonylamino, benzyloxycarbonylamino, cyclohexylamino, 1,3-butanediol, 2-methyl-1,3-butanediol, 1,3-pentanediol, and 2,4-pentanediol;
R₃₁ is selected from the group consisting of hydrogen H, fluorine F, chlorine Cl, bromine Br, iodine I, hydroxyl OH, amino NH2, hydrazino NHNH2, thiol SH, trifluoromethanesulfonyloxy, methanesulfonyloxy, benzenesulfonyloxy, para-toluenesulfonyloxy, para-fluorobenzenesulfonyloxy, ortho-nitrobenzenesulfonyloxy, para-nitrobenzenesulfonyloxy, acetyloxy, trifluoroacetyloxy, benzoyloxy, pivaloyloxy, benzyloxy, para-methoxybenzyloxy, 3,4-dimethoxybenzyloxy, triphenylmethoxy, 2-tetrahydropyranyloxy, methoxymethoxy, 2-ethoxyethoxy, 2-(trimethylsilyl)ethoxymethoxy, allyloxy, benzyloxycarbonyloxy, 4-nitrobenzyloxycarbonyloxy, pentafluorobenzyloxycarbonyloxy, trimethylsiloxy, tert-butyldimethylsiloxy, tert-butyldiphenylsiloxy, diethylisopropylsiloxy, triisopropylsiloxy, triphenylsiloxy, trifluoromethanesulfonylamino, methanesulfonylamino, benzenesulfonylamino, para-toluenesulfonylamino, para-fluorobenzenesulfonylamino, ortho-nitrobenzenesulfonylamino, para-nitrobenzenesulfonylamino, 2-(trimethylsilyl)ethylsulfonyl, acetylamino, trifluoroacetylamino, benzoylamino, pivaloylamino, ortho-tolylureido, benzylamino, para-methoxybenzylamino, 3,4-dimethoxybenzylamino, allylamino, triphenylamino, methoxycarbonylamino, ethoxycarbonylamino, benzyloxycarbonylamino, tert-butyloxycarbonylamino, fluorenyloxycarbonylamino, allyloxycarbonylamino, trimethylsiloxyethoxycarbonylamino, and trichloroethoxycarbonylamino;
R₃₂ is a ketone protecting group derived from methanol, ethanol, 1-propanol, 1-butanol, 1-pentanol, ethylene glycol, 1,2-propanediol, 1,3-propanediol, 2-methyl-1,3-propanediol, 2-ethyl-1,3-propanediol, 2-n-propyl-1,3-propanediol, 2-ethyl-2-methyl-1,3-propanediol, 2-isopropylpropane-1,3-diol, 2-tert-butylpropane-1,3-diol, 2-methoxy-1,3-propanediol, 2,2-dimethyl-1,3-propanediol, 2,2-ethyl-1,3-propanediol, 1,1-cyclohexanedimethanol, 1,1-bis(hydroxymethyl)cyclobutane, 1,1-bis(hydroxymethyl)cyclopentane, 1,1-bis(hydroxymethyl)cyclohexane, 1,3-butanediol, 2-methyl-1,3-butanediol, 1,3-pentanediol, or 2,4-pentanediol; and
R₃₃, R₃₄, R₃₅, R₃₆, and R₃₇ is hydrogen H, methyl, ethyl, propyl, isopropyl, tert-butyl, benzyl, allyl, para-methoxybenzyl, 3,4-dimethoxybenzyl, or triphenylmethyl, respectively.

8. The method according to claim 7, wherein the compound V has the following structure: 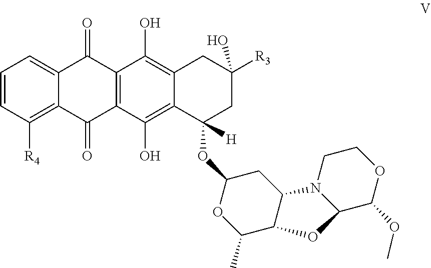
wherein 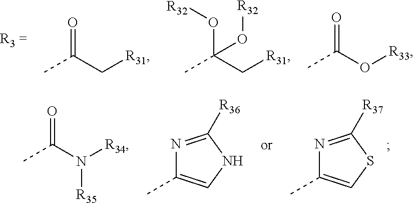
R₄ is selected from the group consisting of hydrogen H, fluorine F, chlorine Cl, bromine Br, iodine I, hydroxyl OH, amino NH2, hydrazino NHNH2, thiol SH, trifluoromethanesulfonyloxy, methanesulfonyloxy, benzenesulfonyloxy, para-toluenesulfonyloxy, para-fluorobenzenesulfonyloxy, ortho-nitrobenzenesulfonyloxy, para-nitrobenzenesulfonyloxy, acetyloxy, trifluoroacetyloxy, benzoyloxy, pivaloyloxy, benzyloxy, para-methoxybenzyloxy, 3,4-dimethoxybenzyloxy, triphenylmethoxy, 2-tetrahydropyranyloxy, methoxymethoxy, 2-ethoxyethoxy, 2-(trimethylsilyl)ethoxymethoxy, allyloxy, benzyloxycarbonyloxy, 4-nitrobenzyloxycarbonyloxy, pentafluorobenzyloxycarbonyloxy, trimethylsiloxy, tert-butyldimethylsiloxy, tert-butyldiphenylsiloxy, diethylisopropylsiloxy, triisopropylsiloxy, triphenylsiloxy, trifluoromethanesulfonylamino, methanesulfonylamino, benzenesulfonylamino, para-toluenesulfonylamino, para-fluorobenzenesulfonylamino, ortho-nitrobenzenesulfonylamino, para-nitrobenzenesulfonylamino, 2-(trimethylsilyl)ethylsulfonyl, acetylamino, trifluoroacetylamino, benzoylamino, pivaloylamino, ortho-tolylureido, benzylamino, para-methoxybenzylamino, 3,4-dimethoxybenzylamino, allylamino, triphenylamino, methoxycarbonylamino, ethoxycarbonylamino, benzyloxycarbonylamino, tert-butyloxycarbonylamino, fluorenyloxycarbonylamino, allyloxycarbonylamino, trimethylsiloxyethoxycarbonylamino, and trichloroethoxycarbonylamino;
R₃₁ is selected from the group consisting of hydrogen H, fluorine F, chlorine Cl, bromine Br, iodine I, hydroxyl OH, amino NH2, hydrazino NHNH2, thiol SH, trifluoromethanesulfonyloxy, methanesulfonyloxy, benzenesulfonyloxy, para-toluenesulfonyloxy, para-fluorobenzenesulfonyloxy, ortho-nitrobenzenesulfonyloxy, para-nitrobenzenesulfonyloxy, acetyloxy, trifluoroacetyloxy, benzoyloxy, pivaloyloxy, benzyloxy, para-methoxybenzyloxy, 3,4-dimethoxybenzyloxy, triphenylmethoxy, 2-tetrahydropyranyloxy, methoxymethoxy, 2-ethoxyethoxy, 2-(trimethylsilyl)ethoxymethoxy, allyloxy, benzyloxycarbonyloxy, 4-nitrobenzyloxycarbonyloxy, pentafluorobenzyloxycarbonyloxy, trimethylsiloxy, tert-butyldimethylsiloxy, tert-butyldiphenylsiloxy, diethylisopropylsiloxy, triisopropylsiloxy, triphenylsiloxy, trifluoromethanesulfonylamino, methanesulfonylamino, benzenesulfonylamino, para-toluenesulfonylamino, para-fluorobenzenesulfonylamino, ortho-nitrobenzenesulfonylamino, para-nitrobenzenesulfonylamino, 2-(trimethylsilyl)ethylsulfonyl, acetylamino, trifluoroacetylamino, benzoylamino, pivaloylamino, ortho-tolylureido, benzylamino, para-methoxybenzylamino, 3,4-dimethoxybenzylamino, allylamino, triphenylamino, methoxycarbonylamino, ethoxycarbonylamino, benzyloxycarbonylamino, tert-butyloxycarbonylamino, fluorenyloxycarbonylamino, allyloxycarbonylamino, trimethylsiloxyethoxycarbonylamino, and trichloroethoxycarbonylamino;
R₃₂ is a ketone protecting group derived from methanol, ethanol, 1-propanol, 1-butanol, 1-pentanol, ethylene glycol, 1,2-propanediol, 1,3-propanediol, 2-methyl-1,3-propanediol, 2-ethyl-1,3-propanediol, 2-n-propyl-1,3-propanediol, 2-ethyl-2-methyl-1,3-propanediol, 2-isopropylpropane-1,3-diol, 2-tert-butylpropane-1,3-diol, 2-methoxy-1,3-propanediol, 2,2-dimethyl-1,3-propanediol, 2,2-ethyl-1,3-propanediol, 1,1-cyclohexanedimethanol, 1,1-bis(hydroxymethyl)cyclobutane, 1,1-bis(hydroxymethyl)cyclopentane, 1,1-bis(hydroxymethyl)cyclohexane, 1,3-butanediol, 2-methyl-1,3-butanediol, 1,3-pentanediol, or 2,4-pentanediol; and
R₃₃, R₃₄, R₃₅, R₃₆, and R₃₇ is hydrogen H, methyl, ethyl, propyl, isopropyl, tert-butyl, benzyl, allyl, para-methoxybenzyl, 3,4-dimethoxybenzyl, or triphenylmethyl, respectively.
